(19) United States
(12) Patent Application Publication
(10) Pub. No.: US 2023/0211085 A1
(43) Pub. Date: Jul. 6, 2023

(54) Autoinjector Housing

(71) Applicant: Alvotech HF, Reykjavik (IS)

(72) Inventors: Elin EDWALD, Reykjavik (IS); Hildur ZOEGA, Reykjavik (IS)

(73) Assignee: Alvotech HF, Reykjavik (IS)

(21) Appl. No.: 18/091,572

(22) Filed: Dec. 30, 2022

Related U.S. Application Data
(60) Provisional application No. 63/295,791, filed on Dec. 31, 2021.
(60) Provisional application No. 63/339,079, filed on May 6, 2022.

(51) Int. Cl. A61M 5/20; A61M 5/32

(52) U.S. Cl. CPC A61M 5/20; A61M 5/3202; A61M 5/322

(57) ABSTRACT

An autoinjector housing includes a cylindrical case enclosing an interior space capable of accommodating an autoinjector mechanism, a cylindrical cap removably attachable to the cylindrical case, and optionally a cylindrical overmolding. An exterior cross-sectional shape of the cylindrical case is oblong along at least a portion of its axis. An exterior cross-sectional shape of the cylindrical cap may be varied along its axis such that there is a gradual decrease in cross-sectional area from an open end toward a closed end and a step-up in cross-sectional area approaching the closed end. An end of the cylindrical case may be covered by the cylindrical overmolding, which may have non-uniform radial thickness and may be made of a material having elasticity.

### CROSS-REFERENCE TO RELATED APPLICATIONS

This application claims priority to U.S. Provisional Application No. 63/295,791, filed Dec. 31, 2021, and U.S. Provisional Application No. 63/339,079, filed May 6, 2022, the disclosures of which are incorporated herein by reference in their entireties.

### BACKGROUND OF THE DISCLOSURE

### Technical Field

The present disclosure relates to an autoinjector housing for an autoinjector used to administer a fluid medicament to a patient.

### Description of the Related Art

An autoinjector is an injection device by means of which a fluid product contained in a product container can be dispensed by a drive mechanism such as a prestressed spring or a gas generator and administered to a patient.

Autoinjectors are described, e.g., in US 2016/0008542 A1. US 2016/0008542 A1 describes an autoinjector, which includes a syringe fixed in a housing. For administration, a needle guard sleeve used as triggering member is set on the desired injection site of the patient. As a result of the autoinjector being pressed against the puncture site, the needle guard sleeve is backed into the housing, wherein, on the one hand, the needle of the syringe is stuck into the patient, and, on the other hand, the drive mechanism is activated, as a result of which the product contained in the product container is expelled by a forward drive member through the needle.

WO 2008/113199 A1 describes an autoinjector, in which the needle guard sleeve is also set on the desired puncture site and backed into the housing by pressing of the autoinjector on the puncture site. As a result, a puncturing sequence is triggered, by means of which the product container is shifted relative to the housing in a direction of an injector end of the housing until the needle protrudes beyond the end of the needle guard sleeve and is stuck into the patient. At the end of the puncturing sequence, a dispensing sequence is triggered, in which a forward drive member expels the product contained in the product container through the needle and injects it into the patient. At the end of the injection process, the autoinjector is removed from the puncture site, as a result of which a retraction sequence is initiated, by means of which the product container is shifted toward the handle end of the housing, as a result of which the needle is retracted into the injector end of the autoinjector, in order to prevent injuries.

In addition, autoinjectors exist that are triggered by shifting of a needle guard sleeve into the housing, wherein the product container can be shifted relative to the housing only for the purpose of the puncturing, wherein the needle remains in the protruding position after the puncturing. For the protection of the needle, the needle guard sleeve is shifted over the needle, and, in particular, locked, in order to prevent a renewed backing of the needle guard sleeve into the housing.

As a result of the design consisting in triggering the puncturing or dispensing by the backing movement of the triggering member, which is often a needle guard sleeve, into the housing, there is a risk that the dispensing of product can be triggered accidentally, e.g., if the autoinjector is dropped or otherwise strongly jolted. In the process, the triggering member can be shifted by its own inertia relative to the housing into its triggering position, as a result of which the dispensing of product is triggered unintentionally.

US 2011/0144594 A1 describes a safety mechanism that is attached at the end opposite that of the needle, i.e., at the handle end, and has to be removed before the triggering of the autoinjector. In order to prepare the autoinjector for dispensing, a covering cap arranged at the injector end of the autoinjector is removed, and the safety mechanism attached at the handle end is removed. As a result of the two steps that have to be carried out separately from one another for preparing the autoinjector, intuitive operation is made more difficult.

While the foregoing autoinjectors represent improvements in safety and ease of patient or caregiver use, in view of the increased reliance on patients to self-administer injectable medicaments in therapeutic regimens (or caregivers to administer in non-clinical environments), there remains a need for autoinjectors that patients or caregivers can intuitively operate at home or in other non-clinical environments safely and without error.

Discussion of references in this application is neither an express nor implied admission of prior art. The disclosures of references identified in this application are expressly incorporated herein by reference in their entireties.

In view of the foregoing, an object of the present disclosure is to provide autoinjector housings, autoinjectors, and methods of using such devices, with advantageous properties.

### BRIEF SUMMARY OF THE DISCLOSURE

According to a first aspect, the present disclosure relates to an autoinjector housing. In embodiments, the autoinjector includes a cylindrical case enclosing an interior space capable of accommodating an autoinjector mechanism, and a cylindrical cap removably attachable to the cylindrical case. The cylindrical case has a first axis extending from a closed handle end to an open injector end of the cylindrical case. An exterior cross-sectional shape of the cylindrical case is varied along the first axis such that there is a step-down in cross-sectional area along the first axis approaching the open injector end. The exterior cross-sectional shape of the cylindrical case is oblong along at least a portion of the first axis. The open injector end of the cylindrical case includes an opening capable of accommodating a retractable needle shield of the autoinjector mechanism. The cylindrical cap has a second axis extending from an open end to a closed end of the cylindrical cap. An exterior cross-sectional shape of the cylindrical cap is varied along the second axis such that there is a gradual decrease in cross-sectional area from the open end toward the closed end and a step-up in cross-sectional area approaching the closed end. The exterior cross-sectional shape of the cylindrical cap is oblong along at least a portion of the second axis. The cylindrical cap is removably attached to the cylindrical case by fitting the open end of the cylindrical cap over the open injector end of the cylindrical case such that the first axis and second axis are substantially aligned. The step-down in cross-sectional area of the cylindrical case forms a cap flange, and attachment of the cylindrical cap is completed when the open end of the cylindrical cap is seated on the cap flange.

According to a second aspect, the present disclosure relates to an autoinjector housing. In embodiments, the autoinjector housing includes a cylindrical case enclosing an interior space capable of accommodating an autoinjector mechanism, a cylindrical cap removably attachable to the cylindrical case, and a cylindrical overmolding. The cylindrical case has a first axis extending from a closed handle end to an open injector end of the cylindrical case. An exterior cross-sectional shape of the cylindrical case is oblong along at least a portion of the first axis. The open injector end of the cylindrical case includes an opening capable of accommodating a retractable needle shield of the autoinjector mechanism. The cylindrical cap has a second axis extending from an open end to a closed end of the cylindrical cap. An exterior cross-sectional shape of the cylindrical cap is oblong along at least a portion of the second axis. The cylindrical cap is removably attached to the cylindrical case by fitting the open end of the cylindrical cap over the open injector end of the cylindrical case such that the first axis and second axis are substantially aligned. The cylindrical overmolding has a third axis extending from a closed end to an open end of the cylindrical overmolding. The cylindrical overmolding encloses an interior space accommodating at least a portion of the cylindrical case such that the third axis coincides with the first axis. At least the closed handle end of the cylindrical case is covered by the cylindrical overmolding. The cylindrical overmolding includes a material having elasticity. The cylindrical overmolding has a non-uniform radial thickness measured in a direction perpendicular to the third axis.

According to a third aspect of the present disclosure, an autoinjector housing as described herein may be included in an autoinjector. In embodiments, such an autoinjector includes an autoinjector housing as described herein and an autoinjector mechanism.

According to a fourth aspect, the present disclosure relates to method of treating at least one disease/disorder selected from the group consisting of rheumatoid arthritis, juvenile idiopathic arthritis, psoriatic arthritis, ankylosing spondylitis, Crohn's disease, ulcerative colitis, plaque psoriasis, hidradenitis suppurativa, and uveitis. In embodiments, the method includes administering a single dose of a tumor necrosis factor (TNF) blocker to a patient in need of such treatment with an autoinjector as described herein.

According to a fifth aspect, the present disclosure relates to a method of injecting a medicament using an autoinjector as described herein. In embodiments, the method includes removing the cylindrical cap from the cylindrical case, if attached, grasping the autoinjector, positioning the autoinjector so a retractable needle shield of the autoinjector is in contact with an injection site on a patient's skin and the first axis of cylindrical case is substantially perpendicular to the injection site, pressing the autoinjector into the skin in a direction substantially perpendicular to the injection site to cause (i) a needle of the autoinjector to insert into the injection site, (ii) the retractable needle shield to retract into the cylindrical case, and (iii) a single dose of the medicament to be injected into the injection site, and pulling the autoinjector away from the injection site resulting in return of the retractable needle shield to an unretracted state when administration of the single dose of the medicament is complete.

The foregoing paragraphs have been provided by way of general introduction and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 is a perspective view of an embodiment of an autoinjector housing according to the present disclosure;

FIG. 2 is a schematic perspective depiction of cylinders or cylindrical objects;

FIG. 3 is a schematic depiction of cross-sections of solid and hollow cylinders or cylindrical objects;

FIG. 4 is a perspective view of an embodiment of an autoinjector housing (without cylindrical cap) according to the present disclosure;

FIG. 5 is a side view of an embodiment of an autoinjector housing (without cylindrical cap) according to the present disclosure;

FIG. 6 is an open injector end view of an embodiment of an autoinjector housing (without cylindrical cap) according to the present disclosure;

FIG. 7 is a top view of an embodiment of an autoinjector housing (without cylindrical cap) according to the present disclosure;

FIG. 8 is a perspective view of an embodiment of a cylindrical cap according to the present disclosure;

FIG. 9 is a closed end view of an embodiment of a cylindrical cap according to the present disclosure;

FIG. 10 is a top view showing the major dimension of an embodiment of a cylindrical cap according to the present disclosure;

FIG. 11 is a side view showing the minor dimension of an embodiment of a cylindrical cap according to the present disclosure;

FIG. 12 is a perspective view of an embodiment of an autoinjector housing according to the present disclosure;

FIG. 13 is a perspective view of an embodiment of a cylindrical overmolding according to the present disclosure;

FIG. 14 is a schematic partial longitudinal section view of an embodiment of an autoinjector housing according to the present disclosure;

FIG. 15 is a schematic partial cross-section view of an embodiment of a cylindrical overmolding according to the present disclosure;

FIG. 16 is a side view of an embodiment of an autoinjector housing according to the present disclosure;

FIG. 17 is a closed end view of an embodiment of a cylindrical overmolding according to the present disclosure; and

FIG. 18 is a schematic partial top view of an embodiment of a cylindrical overmolding according to the present disclosure.

### DETAILED DESCRIPTION OF THE EMBODIMENTS

Embodiments of the present disclosure will now be described more fully hereinafter with reference to the accompanying drawings, in which some, but not all of the embodiments of the disclosure are shown.

As used herein, the words “a” and “an” and the like carry the meaning of “one or more.” Additionally, within the description of this disclosure, where a numerical limit or range is stated, the endpoints are included unless stated otherwise. Also, all values and subranges within a numerical limit or range, including subranges excluding endpoints, are specifically included as if explicitly written out.

As used herein, the terms “optional” or “optionally” mean that the subsequently described event(s) can or cannot occur or the subsequently described component(s) may or may not be present (e.g., 0 wt %).

The term “comprising” is an open-ended term synonymous with terms such as including, containing, or having and is used herein to claim aspects of the invention which may include additional steps, components, functionality, and/or structure. The term “consisting essentially of” is used herein to claim aspects of the invention and additional unrecited steps or components that would not have a material effect on the basic and novel properties of an autoinjector housing, autoinjector, or method according to the present disclosure. Basic and novel properties of the present disclosure include improvements in intuitive operation, safe operation, and effective administration of a dose of a liquid medicament during the course of self-injection by a patient or injection by a caregiver in a non-clinical environment, relative to known autoinjectors. The term “consisting of” is used to claim aspects of the invention in which only those features explicitly recited in a claim are included and thus other steps or components not explicitly or inherently included in the claim are excluded.

It is expressly intended that the features of the various features of autoinjector housings and autoinjectors described herein be interchangeably used and used in combination. That is, to the extent that a material, structure, and/or size is mentioned with respect to an embodiment, it is expressly intended that such parameter be suitably substituted for use in other embodiments. The present inventors have contemplated such combinations and intend that they be encompassed by this disclosure even if not written out.

Autoinjector housings and components thereof described herein may have any suitable combination of dimensions, and the dimensions of particular embodiments are selected to produce desired effects described herein in combination with particular materials and other structural parameters. Autoinjector housings and components thereof described herein may be described in terms of the dimensions and ranges of dimensions explicitly set forth herein, as well as any ratios thereof. If an average dimension is referenced, the average may be calculated using a reasonable number, e.g., five or ten, of measurements as a basis for the average.

The autoinjector housing according to the present disclosure, when paired with an appropriate autoinjector mechanism, provides numerous advantages relative to known autoinjector housings. The autoinjector housing is designed for ease-of-use generally and by dexterity-impaired patients or caregivers. The autoinjector housing is designed for two-step injection (remove the cap and press into injection site), which reduces avenues for injury and errors in injection. The autoinjector housing has a wide (oblong) cross-section, which is easy to grasp and hold, and resists rolling when laid, e.g., on a table. In embodiments, the autoinjector housing is of a modest length compared with known autoinjector housings, facilitating ease of handling. In embodiments, the autoinjector housing has a single cap and a grip (overmolding), which make handling easy, and make the directionality of the autoinjector housing (i.e., the handle end and the injector end) more intuitive and, thus, less prone to injury and errors in injection. In embodiments, the grip is made of a soft, rubbery material having openings that facilitate a comfortable stable grasp and make it easy to recognize the appropriate location to grip. In embodiments, the cap has a body that tapers in width and then transitions to a widened edge at its closed end, which make identification and removal of the cap easier. In embodiments, the autoinjector housing is provided with a color/textural scheme that allows a patient or caregiver to intuitively perceive the function of respective parts of the autoinjector housing.

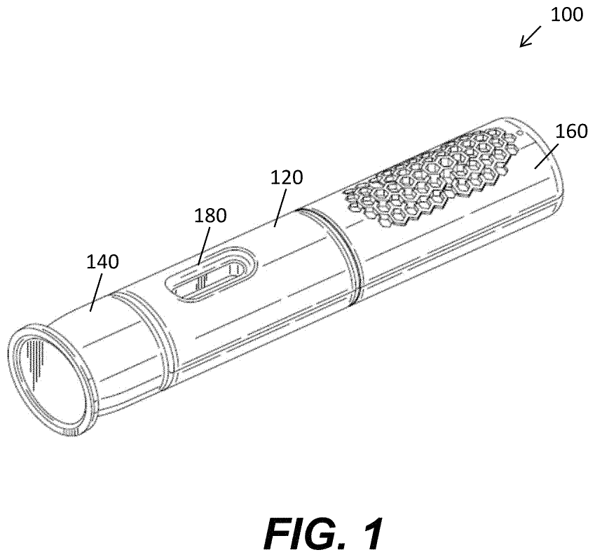

An exemplary embodiment of an autoinjector housing according to the present disclosure is shown in FIG. 1. As shown in FIG. 1, an autoinjector housing 100 includes a cylindrical case 120 enclosing an interior space capable of accommodating an autoinjector mechanism (not shown), and a cylindrical cap 140 removably attachable to the cylindrical case. In embodiments, the autoinjector housing 100 further includes a cylindrical overmolding 160 covering a portion of the cylindrical case 120 and/or a window opening 180 formed in the cylindrical case 120.

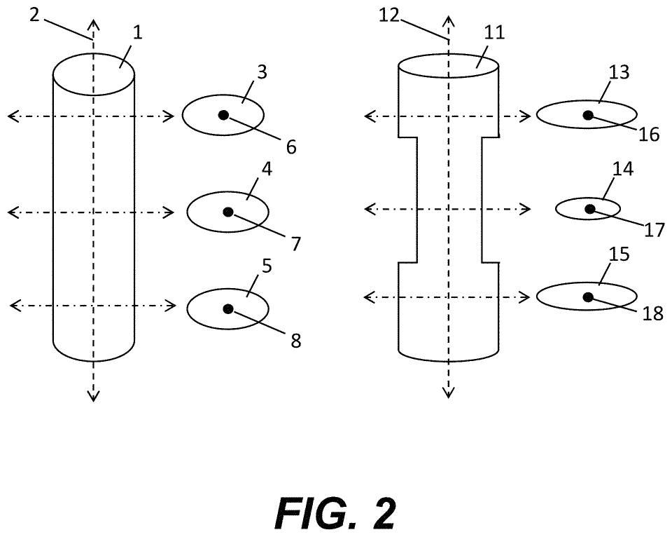

The terms “cylinder” and “cylindrical” are used throughout this application. It is expressly intended that such terms not be limited to their strict mathematical/geometric definitions. While a cylinder is generally a solid object with identical flat ends that are circular or elliptical and one curved side, cylinders and cylindrical objects as described herein are not so limited. The objects described as cylinder and cylindrical are non-spherical and have an axis that is also the geometric center of all cross sections of the object that are perpendicular to the axis. Even in this regard, the axis need not be the precise geometric center of each cross-section as some cylinders or cylindrical objects may have surface projections or depressions that result in variations from true cylindrical symmetry. FIG. 2 is a schematic depiction of two cylinders or cylindrical objects 1 or 11. Each of the objects 1 or 11 has an axis 2 or 12, and each cross-section 3, 4, 5 or 13, 14, 15 is perpendicular to the axis 2 or 12 has a geometric center 6, 7, 8 or 16, 17, 18 that corresponds to the axis 2 or 12. In FIG. 2, the cross-sections 3, 4, 5 are circles (shown in perspective), but they need not be (compare cross-sections 13, 14, 15, which are ellipses). Particularly, autoinjectors described herein may have an oblong exterior cross-sectional shape along some portion of their length. Further, in FIG. 2, the cross-sections 3, 4, 5 have the same cross-sectional area, but they need not (compare cross-sections 13, 14, 15, which have varied cross-sectional areas). Objects are often described herein with respect to variations in cross-sectional shape or area along an object's axis. If the cylinder or cylindrical object 1 as described herein was, e.g., a truncated cone, the cross-sections 3, 4, 5 would be circles, but they would sequentially increase (or decrease) in area along the axis 2. Alternatively, as in the cylinder or cylindrical object 11, the cross-sections 13, 14, 15 vary irregularly in area along the axis 12.

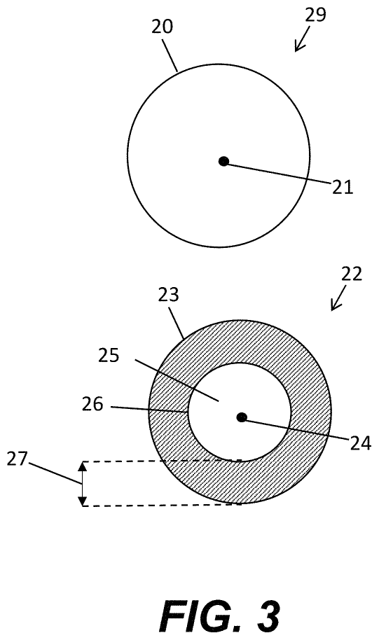

As used herein, “exterior cross-sectional shape” refers to the shape defined by the outermost perimeter of any cross-section along the axis of an object. As used herein, “cross-sectional area” refers to the area enclosed by the “exterior cross-sectional shape.” Two cross-sections might have identical “exterior cross-sectional shapes,” but may have different interior cross-sectional shapes—to provide a hollow space for accommodating the autoinjector, projections and/or recesses for attachment to the autoinjector mechanism, etc. Because “cross-sectional area” is defined as the entire area enclosed by the “exterior cross-sectional shape” (and not the actual area enclosed by the exterior cross-sectional shape and the interior cross-sectional shape), unless otherwise specified, two cross-sections having identical “exterior cross-sectional shapes,” but different interior cross-sectional shapes will be deemed to have the same “cross-sectional area,” notwithstanding the differences in interior features. In FIG. 3, a cross-section 29 of a solid object has an exterior cross-sectional shape 20, which is its outermost perimeter; the axis 21 of the solid object is also shown. A cross-section 22 of a hollow object has an exterior cross-sectional shape 23, which is also its outermost perimeter; the axis 24 of the hollow object is also shown (the solid portion of the hollow object is shown with cross-hatching). A hollow space 25 is enclosed by an interior cross-sectional shape 26. As used herein, the cross-sectional areas of the respective cross-sections 29, 22 are the same.

The hollow object in FIG. 3 may be defined at any location around its periphery by its radial thickness 27. The radial thickness is the difference between the maximum and minimum distances of the hollow object from the axis 24 at a given location along the interior and exterior periphery of the hollow object.

Exterior cross-sectional shapes are variously described as oblong, oval, elliptical, and polygonal. As noted above, cylinders and cylindrical objects described herein need not have geometrically circular or geometrically elliptical exterior cross-sectional shapes. An oblong shape is an elongated shape, i.e., a shape having a major dimension and a minor dimension. An oblong shape can have a wholly curved perimeter, a polygonal perimeter, some combination thereof. Ovals, ellipses, and rectangles are examples of oblong shapes. An oval is an elongated shape having a wholly curved perimeter. An ellipse is an example of an oval. An ellipse or elliptical shape is an elongated shape having a wholly curved perimeter in which the major axis (longest dimension) and minor axis (smallest dimension) are perpendicular and bisect one another. Although a mathematically-defined ellipse is included withing this meaning of ellipse or elliptical, the terms as used herein are not so limited. A polygon or polygonal shape is a shape having only straight lines and vertices along its perimeter. Polygons include triangles, squares and other four-sided shapes, pentagons, hexagons, octagons, etc. Each of the above shapes as used herein are permissive of variations in perimeter to accommodate features of an autoinjector housing. A cutout in an elliptical cross-section of the cylindrical case 120 to provide the window opening 180, e.g., as in FIG. 1, or a projection or recess on an elliptical cross-section of the cylindrical overmolding 160 to provide a texture for grip would not prevent such cross-sections from being deemed elliptical.

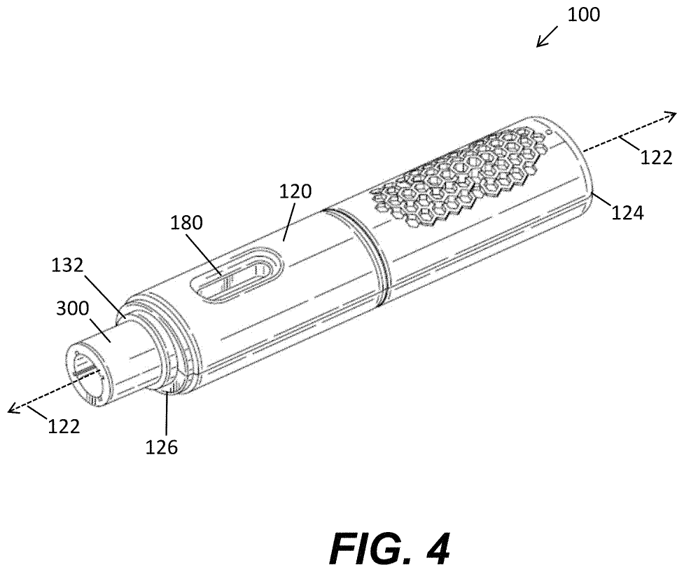

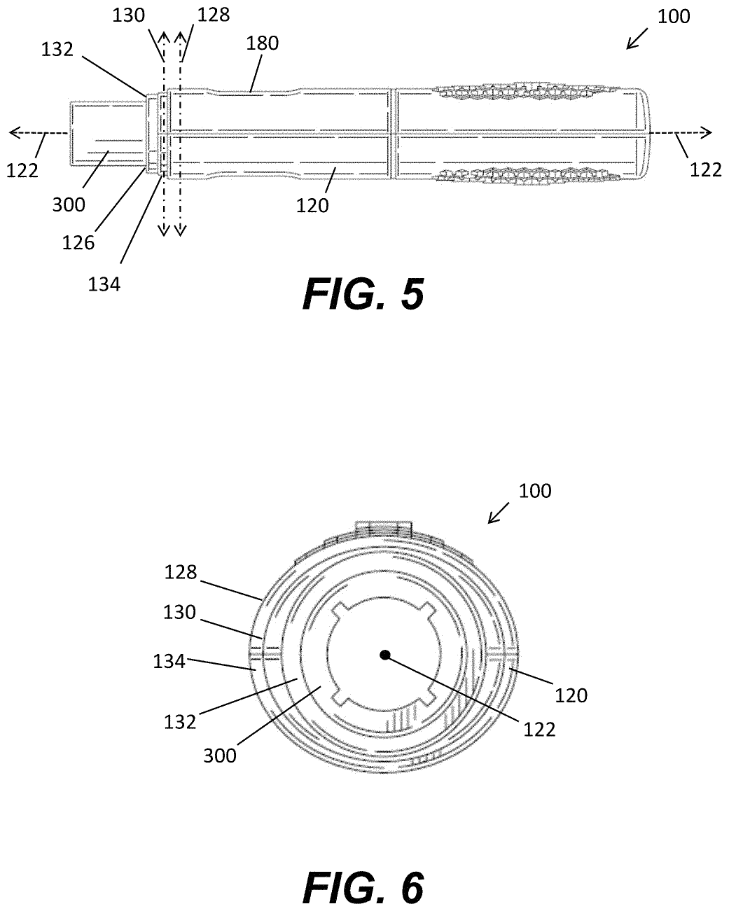

As shown in FIG. 4, the cylindrical case 120 has a first axis 122 extending from a closed handle end 124 to an open injector end 126 of the cylindrical case 120. Of course, the first axis 122 is not an actual feature of the cylindrical case 120, but rather a vector of reference used to describe various features of the cylindrical case 120. As shown in FIGS. 5 and 6, an exterior cross-sectional shape of the cylindrical case 120 is varied along the first axis 122 such that there is a step-down in cross-sectional area along the first axis 122 approaching the open injector end 126. An example of the step-down in cross-sectional area is shown with reference to the first cross-section 128 and the second cross-section 130 shown in FIGS. 5 and 6. The exterior cross-sectional shape of the cylindrical case 120 is oblong along at least a portion of the first axis. As is particularly evident in FIG. 6, which is a view perpendicular to the first axis 122, the first cross-section 128 and the second cross-section 130 are examples of locations along the first axis 122 where an exterior cross-sectional shape of the cylindrical case 120 is oblong and, particularly, elliptical. As shown in FIGS. 4-6, the open injector end 126 of the cylindrical case comprises an opening 132 capable of accommodating a retractable needle shield 300 of the autoinjector mechanism.

A length of the cylindrical case 120 along the first axis 122 from the closed handle end 124 to the open injector end 126 may preferably be: (i) greater than or equal to 110 mm and less than or equal to 140 mm, (ii) greater than or equal to 125 mm and less than or equal to 130 mm, (iii) any combination of greater than or equal to 110 mm, 115 mm, 120 mm, or 125 mm, and less than or equal to 130 mm, 135 mm, or 140 mm, or (iv) even more preferably 126 mm or about 126 mm.

The autoinjector housing 100 as described herein is held in a patient or caregiver's hand during use. Measurements of adult hand size may include length, which may be measured from the tip of the longest finger to the crease under the palm, and breadth, which may be measured across the widest area where the fingers join the palm. According to the National Aeronautics and Space Administration (NASA), American males have an average hand length of 7.6 inches (193 mm) and an average hand breadth of 3.5 inches (89 mm). American females have an average hand length of 6.8 inches (173 mm) and an average hand breadth of 3.1 inches (79 mm).

Because the cylindrical case 120, and thus the autoinjector housing 100, is of modest length compared to known autoinjector housings, it fits more firmly within the grasp of most patients or caregivers and thus feels more stably grasped. The ends of the autoinjector housing 100 do not protrude greatly beyond the edges of a patient or caregiver's hand. This reduces the likelihood that the autoinjector housing 100 will be dropped or mishandled during injection causing inconvenience and/or damage to its contents. A ratio of hand length to the length of the cylindrical case 120 along the first axis from the closed handle end 124 to the open injector end 126, for men, may preferably be: (i) greater than or equal to 1.3 and less than or equal to 1.75, (ii) greater than or equal to 1.5 and less than or equal to 1.3, or (iii) even more preferably 1.5 or about 1.5. A ratio of hand length to the length of the cylindrical case 120 along the first axis from the closed handle end 124 to the open injector end 126, for women, may preferably be: (i) greater than or equal to 1.25 and less than or equal to 1.55, (ii) greater than or equal to 1.25 and less than or equal to 1.35, or (iii) even more preferably 1.35 or about 1.35. A ratio of hand breadth to the length of the cylindrical case 120 along the first axis from the closed handle end 124 to the open injector end 126, for men, may preferably be: (i) greater than or equal to 0.6 and less than or equal to 0.8, (ii) greater than or equal to 0.6 and less than or equal to 0.7, or (iii) even more preferably 0.7 or about 0.7. A ratio of hand breadth to the length of the cylindrical case 120 along the first axis from the closed handle end 124 to the open injector end 126, for women, may preferably be: (i) greater than or equal to 0.5 and less than or equal to 0.7, (ii) greater than or equal to 0.5 and less than or equal to 0.6, or (iii) even more preferably 0.6 or about 0.6.

An average major dimension of the exterior cross-sectional shape of the cylindrical case 120 along the first axis 122 from the closed handle end 124 to the open injector end 126 may preferably be: (i) greater than or equal to 20 mm and less than or equal to 40 mm, (ii) greater than or equal to 25 mm and less than or equal to 30 mm, (iii) any combination of greater than or equal to 20 mm, or 25 mm, and less than or equal to 30 mm, 35 mm, or 40 mm, or (iv) even more preferably 28 mm or about 28 mm. The foregoing major dimensions are desirable even in a cylindrical case 120 that does not have an oblong exterior cross-sectional shape along at least a portion of its first axis 122 but is otherwise constructed as described herein. That is, the present disclosure encompasses an autoinjector housing 100 including a cylindrical case 120 that has any exterior cross-sectional shape with a largest dimension that is at least any of the lengths described in this paragraph, with the understanding that grasping such an autoinjector housing 100 would be easier for patients and or caregivers that struggle with dexterity and strength, relative to known autoinjector housings.

An average minor dimension of the exterior cross-sectional shape of the cylindrical case 120 along the first axis 122 from the closed handle end 124 to the open injector end 126 may preferably be (i) greater than or equal to 15 mm and less than or equal to 35 mm, (ii) greater than or equal to 20 mm and less than or equal to 25 mm, (iii) any combination of greater than or equal to 15 mm or 20 mm, and less than or equal to 25 mm, 30 mm, or 35 mm, or (iv) even more preferably 23 mm or about 23 mm.

Because the cylindrical case 120 has an oblong exterior cross-sectional shape along at least portions of its length, the autoinjector housing 100 is less likely to freely roll or rotate, either when laid on a flat surface like a table or when in a patient or caregiver's hand. For this additional reason, the autoinjector fits more firmly within the grasp of most patients or caregivers, giving a feeling of stability, and the autoinjector housing 100 is less likely to roll away when preparing for an injection. This, again, reduces the likelihood that the autoinjector housing 100 will be dropped causing inconvenience and/or damage to its contents. A ratio of hand length to an average major dimension of the exterior cross-sectional shape of the cylindrical case 120 along the first axis 122 from the closed handle end 124 to the open injector end 126, for men, may preferably be: (i) greater than or equal to 4.8 and less than or equal to 9.6, (ii) greater than or equal to 4.8 and less than or equal to 6.9, or (iii) even more preferably 6.9 or about 6.9. A ratio of hand length to an average major dimension of the exterior cross-sectional shape of the cylindrical case 120 along the first axis 122 from the closed handle end 124 to the open injector end 126, for women, may preferably be: (i) greater than or equal to 4.3 and less than or equal to 8.7, (ii) greater than or equal to 4.3 and less than or equal to 6.2, or (iii) even more preferably 6.2 or about 6.2.

In embodiments, the cylindrical case 120 is formed by molding a polymeric material. In embodiments, the cylindrical case 120 is formed by molding a thermoplastic polymeric material. In embodiments, the cylindrical case 120 is formed of a polymeric material that, after molding and post-molding processing, is rigid. In embodiments, the cylindrical case 120 is formed of a polymeric material that, after molding and processing, is opaque and colored. In embodiments, the polymeric material is white. In embodiments, an exterior surface of the cylindrical case 120 has printed subject matter (e.g., branding, usage instructions, etc.) formed thereon. In embodiments, the cylindrical case 120 is formed of a PC/ABS (a polycarbonate/acrylonitrile-butadiene-styrene terpolymer blend). In alternative embodiments, the cylindrical cap 140 is formed of an acrylonitrile butadiene styrene, a polycarbonate, an aliphatic polyamide, a polyoxymethene, a polymethyl methacrylate, a polypropylene, a polybutylene terephthalate, a polyphenyl sulfone, a polyether ether ketone, or a polyetherimide.

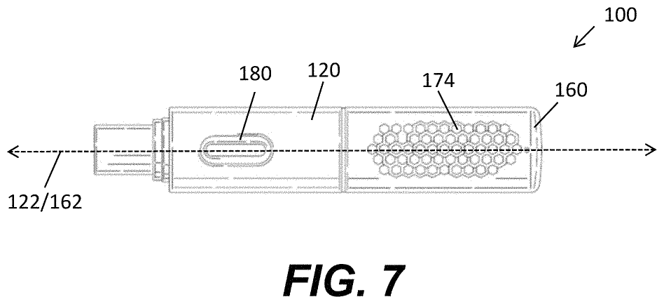

As shown in FIGS. 4 and 5, in embodiments, the cylindrical case 120 includes at least one window opening 180 extending through the cylindrical case 120 in a radial direction. When the autoinjector mechanism is accommodated in the interior space of the cylindrical case 120 and includes a syringe, contents of the syringe (and hence the used/unused status of the autoinjector) can be seen through the window opening 180. As shown in FIG. 7, the window opening 180 has an oblong shape when viewed from an exterior of the cylindrical case 120 with a major axis of the oblong shape being parallel to the first axis 122 of the cylindrical case 120. A length of a major axis of the of the window opening 180 may preferably be (i) greater than or equal to 15 mm and less than or equal to 35 mm, (ii) greater than or equal to 20 mm and less than or equal to 30 mm, (iii) any combination of greater than or equal to 15 mm or 20 mm, and less than or equal to 30 mm, or 35 mm, or (iv) even more preferably 20 mm, about 20 mm, 27 mm, or about 27 mm. A length of a minor axis of the of the window opening 180 may preferably be (i) greater than or equal to 3 mm and less than or equal to 9 mm, (ii) greater than or equal to 3 mm and less than or equal to 7 mm, or (iii) even more preferably 5 mm or about 5 mm.

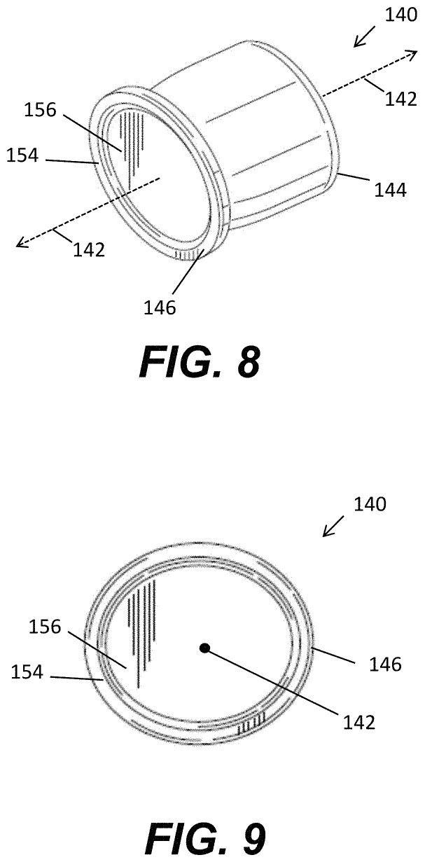

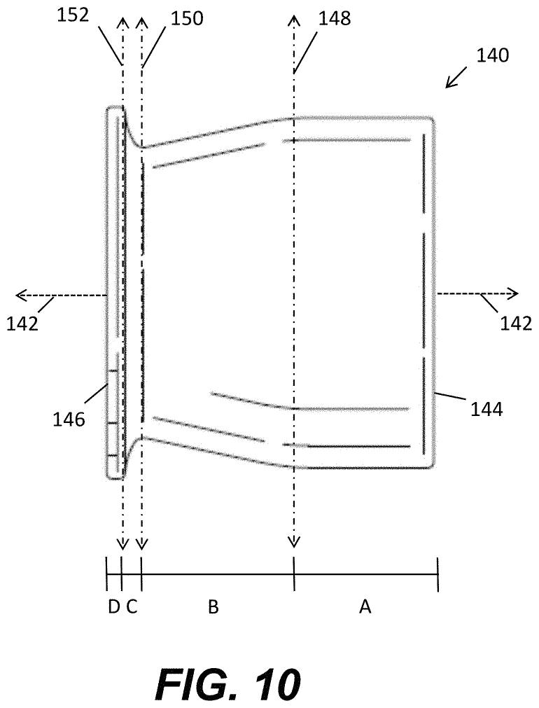

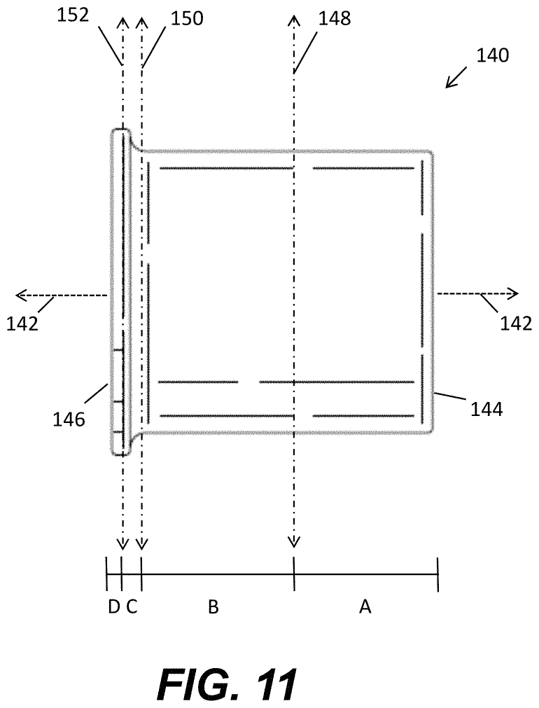

As shown in FIG. 8, the cylindrical cap 140 has a second axis 142 extending from an open end 144 to a closed end 146 of the cylindrical cap 140. As can be seen from the closed end 144 of the cylindrical cap 140 shown in FIG. 9, the exterior cross-sectional shape of the cylindrical cap 140 is oblong, and particularly elliptical, along at least a portion of the second axis 142. As shown in FIGS. 10 and 11, the exterior cross-sectional shape of the cylindrical cap 140 is varied along the second axis 142 such that there is a gradual decrease in cross-sectional area from the open end 144 toward the closed end 146 (Section B) and a step-up in cross-sectional area approaching the closed end 146 (Section C). Viewing FIG. 10, the gradual decrease in cross-sectional area can be seen in the transition from the first cross-section 148 to the second cross-section 150 (Section B). The step-up in cross-sectional area can be seen in the transition from the second cross-section 150 to the third cross-section 152 (Section C). The gradual decrease in cross-sectional area is not seen in the transition from the first cross-section 148 to the second cross-section 150 in the side view of FIG. 11, because, along the second axis 142, the minor axis of the exterior cross-sectional shape of the cylindrical cap 140 remains substantially the same from the open end 144 to the step-up in cross-sectional area.

Referring to FIGS. 10 and 11, in embodiments, the cylindrical cap 140 includes a first section A, a second section B, and a third section C, and a fourth section D, in order, along the second axis 142 from the open end 144 to the closed end 146. The first section A extends from the open end 144 to the first cross-section 148; the second section B extends from the first cross-section 148 to the second cross-section 150; the third section C extends from the second cross-section 150 to the third cross-section 152; and the fourth section D extends from the third-cross section 152 to the closed end 146. The cross-sectional area is substantially constant in the first section A. The gradual decrease in cross-sectional area along the second axis 142 from the open 144 end toward the closed end 146 of the cylindrical cap 140 occurs in the second section B (resulting from the gradual decrease in the length of the major axis of the exterior cross-sectional shape along the second axis 142 approaching the second cross-section 150). The step-up in cross-sectional area along the second axis 142 approaching the closed end 146 of the cylindrical cap 140 occurs in the third section C. The exterior cross-sectional area of the cylindrical cap 140 along the second axis 142 is greater in the fourth section D than at any point along the second axis 142 in the first section A, second section B, or third section C. The exterior cross-sectional shape of the cylindrical cap 140 along the second axis 142 in the first section A and the second section B is oblong. The length of the major axis of the oblong exterior cross-sectional shape of the cylindrical cap 140 gradually decreases along the second axis 142 in the second section B until the second cross-section 150 such that the lengths of the major axis and the minor axis are the same or approximately the same at the second cross-section 150. That is, at second cross-section 150, the exterior cross-sectional shape of the cylindrical cap 140 is circular or approximately circular. The fourth section D of the cylindrical cap 140 constitutes less than one tenth of a length of the cylindrical cap 140 along the second axis 142.

As shown in FIGS. 8 and 9, in embodiments, the closed end 146 of the cylindrical cap 140 comprises a standing surface 154 substantially perpendicular to the second axis 142. When the cylindrical cap 140 is attached to the cylindrical case 120, the autoinjector housing 100 can be stably stood on a flat surface by placing the standing surface 154 in contact with the flat surface. In embodiments, the standing surface 154 of the closed end 146 of the cylindrical cap 140 is provided at a periphery of the closed end 146 and surrounds an interior surface 156 that is recessed with respect to the standing surface 146. In embodiments, the interior surface 156 comprises more than 75 percent of a surface area of the cylindrical cap 140 when viewing the closed end 146 of the cylindrical cap (i.e., in view shown in FIG. 9).

In embodiments, the cylindrical cap 140 includes an interior space that accommodates the open injector end 126 of the cylindrical case 120 when the cylindrical cap 140 is attached to the cylindrical case 120. The interior space of the cylindrical cap 140 may include a needle sheath for receiving the retractable needle shield 300 when present. The interior space of the cylindrical cap 140 may further include claw grabbers for engaging with the autoinjector mechanism when present.

In embodiments, the cylindrical cap 140 is the only cap of the autoinjector housing 100. That is, prior to use of an autoinjector including the autoinjector housing 100, a patient or caregiver need only remove the single cylindrical cap 140 in order to place the autoinjector in condition for use. In embodiments, the autoinjector housing 100 does not include an opening for accommodating a button or actuator. That is, an autoinjector including the autoinjector housing 100 does not have a button or actuator that must be pressed by the patient or caregiver during use.

In embodiments, the cylindrical cap 140 is made of a transparent or translucent material.

A length of the cylindrical cap 140 along the second axis 142 from the open end 144 to the closed end 146 may preferably be (i) greater than or equal to 15 mm and less than or equal to 35 mm, (ii) greater than or equal to 25 mm and less than or equal to 30 mm, (iii) any combination of greater than or equal to 15 mm, 20 mm, or 25 mm, and less than or equal to 30 mm or 35 mm, or (iv) even more preferably 26 mm or about 26 mm.

A major dimension of the exterior cross-sectional shape of the cylindrical cap 140 at the open end 144 may preferably be (i) greater than or equal to 20 mm and less than or equal to 40 mm, (ii) greater than or equal to 26 mm and less than or equal to 30 mm, (iii) any combination of greater than or equal to 20 mm or 25 mm, and less than or equal to 30 mm, 35 mm, or 40 mm, or (iv) even more preferably 28 mm or about 28 mm.

A minor dimension of the exterior cross-sectional shape of the cylindrical cap 140 at the open end 144 may preferably be (i) greater than or equal to 15 mm and less than or equal to 35 mm, (ii) greater than or equal to 21 mm and less than or equal to 25 mm, (iii) any combination of greater than or equal to 15 mm or 20 mm, and less than or equal to 25 mm, 30 mm, or 35 mm, or (iv) even more preferably 23 mm or about 23 mm.

A major dimension of the exterior cross-sectional shape of the cylindrical cap 140 at the closed end 146 may preferably be (i) greater than or equal to 20 mm and less than or equal to 40 mm, (ii) greater than or equal to 28 mm and less than or equal to 32 mm, (iii) any combination of greater than or equal to 20 mm or 25 mm, and less than or equal to 35 mm, or 40 mm, or (iv) even more preferably 30 mm or about 30 mm.

In embodiments, a minor dimension of the exterior cross-sectional shape of the cylindrical cap 140 at the closed end 146 may preferably be (i) greater than or equal to 15 mm and less than or equal to 35 mm, (ii) greater than or equal to 25 mm and less than or equal to 29 mm, (iii) any combination of greater than or equal to 15 mm, 20 mm, or 25 mm, and less than or equal to 30 mm, or 35 mm, or (iv) even more preferably 27 mm or about 27 mm.

A distance from the step-up in cross-sectional area of the exterior cross-sectional shape of the cylindrical cap 140 along the second axis 142 approaching the closed end 146 to the closed end 147 may preferably be (i) greater than or equal to 1 mm and less than or equal to 5 mm, (ii) any combination of greater than or equal to 1 mm, and less than or equal to 2 mm, 3 mm, 4 mm, or 5 mm, or (iv) even more preferably less than 3 mm or less than 2.5 mm.

The size and shape of the cylindrical cap 140 provide distinct advantages over caps in known autoinjector housings. Referring to FIGS. 10 and 11, proceeding from the open end 144 to the closed end 146 from a perspective along the minor axis, in embodiments, the cross-section of the cylindrical cap 140 is first oblong (and the same as the cylindrical case 120 so there is a smooth dimensional transition where the two join when the cylindrical cap 140 is attached (Section A), then gradually becomes a smaller and nearly circular neck (Section B), and then finally quickly returns to an oblong shape (Section C) even larger than at the open end 144 to form a rim (Section D). This rim makes it easy to grip the cylindrical cap 140 when removing from the cylindrical case 120. Further, because of the dimensions above and as evident, e.g., in FIGS. 10 and 11, the increased width of the rim is not merely cosmetic, but provides mechanical advantage in removing the cap. Moreover, the size and location of the rim cue to a user that the cylindrical cap 140 is, in fact, a cap and not some other part. In addition, as discussed above with respect to the standing surface 154 and the interior surface 156 of the closed end 146 of the cylindrical cap 140, the size and shape of the cylindrical cap make it possible to stably stand the autoinjector housing 100 on its end, e.g., on a table. This makes it easier to view the autoinjector from a seated position, allowing the patient or caregiver to view the contents of the autoinjector housing 100 (e.g., through the window opening 180) and any instructions or indications printed on the cylindrical case 120 without picking up or leaning over the autoinjector housing 100. This can be particularly desirable when a patient or caregiver is not yet ready to handle the autoinjector housing 100 while, e.g., reading instructions, cleaning the injection site, etc.

In embodiments, the cylindrical cap 140 is formed by molding a polymeric material. In embodiments, the cylindrical cap 140 is formed of a polymeric material that, after molding and post-molding processing, is rigid. In embodiments, the cylindrical cap 140 is formed of a polymeric material that, after molding and processing, is transparent or translucent. In embodiments, the cylindrical cap 140 is formed of a polycarbonate. In alternative embodiments, the cylindrical cap 140 is formed of a polycarbonate/acrylonitrile-butadiene-styrene terpolymer blend, an acrylonitrile butadiene styrene, an aliphatic polyamide, a polyoxymethene, a polymethyl methacrylate, a polypropylene, a polybutylene terephthalate, a polyphenyl sulfone, a polyether ether ketone, or a polyetherimide.

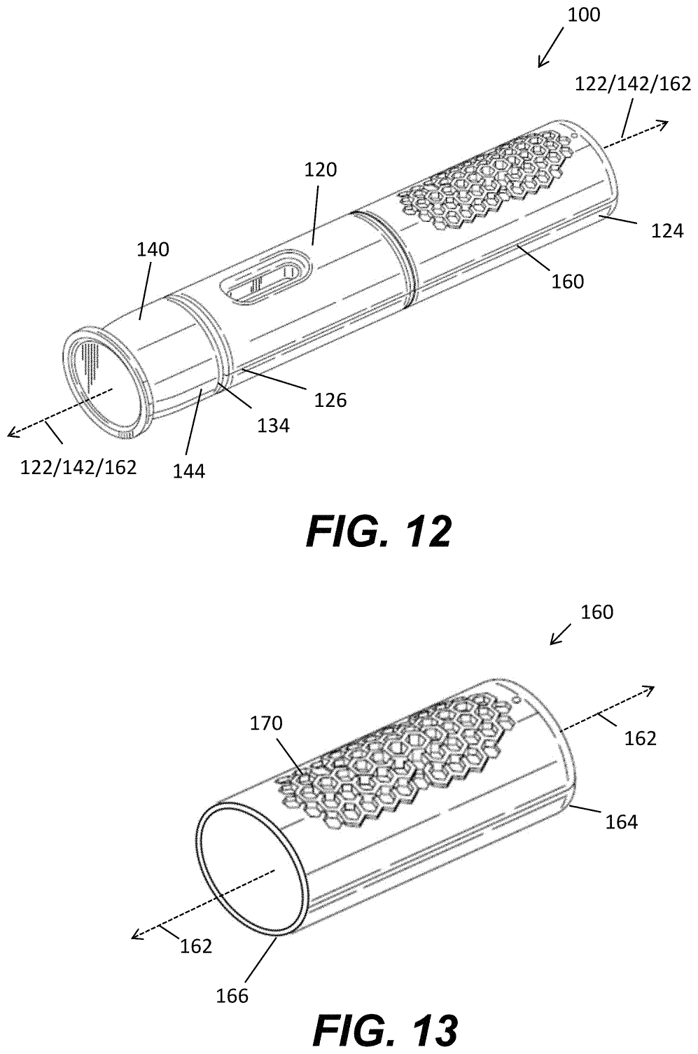

As shown in FIG. 12, the cylindrical cap 140 is removably attached to the cylindrical case 120 by fitting the open end 144 of the cylindrical cap 140 over the open injector end 126 of the cylindrical case 120 such that the first axis 122 and second axis 142 are substantially aligned. The step-down in cross-sectional area of the cylindrical case 120 along the first axis 122 approaching the open injector end 126 (i.e., transition from to the first cross-section 128 to the second cross-section 130 shown in FIGS. 5 and 6) forms a cap flange 134 and attachment of the cylindrical cap 140 is completed when the open end 144 of the cylindrical cap 140 is seated on the cap flange 134.

A total length of the autoinjector housing 100, with the cylindrical cap 140 attached to the cylindrical case 120, along the first axis 122 and coincident second axis 142 may preferably be (i) greater than or equal to 135 mm and less than or equal to 165 mm, (ii) greater than or equal to 145 mm and less than or equal to 155 mm, (iii) any combination of greater than or equal to 135 mm, 140 mm, or 145 mm, and less than or equal to 150 mm, 155 mm, 160 mm, or 165 mm, or (iv) even more preferably 150 mm or about 150 mm. Even when the cylindrical cap 140 is attached to the cylindrical case 120, the length remains modest. Thus, it is more easily gripped, reducing the likelihood that the autoinjector housing 100 will be dropped causing inconvenience to a patient or caregiver and/or damage to its contents.

In embodiments, the oblong exterior cross-sectional shape of the cylindrical case 120 along at least a portion of the first axis 122 is selected from the group consisting of substantially oval, substantially hexagonal, substantially octagonal, and substantially polygonal. In embodiments, the oblong exterior cross-sectional shape of the cylindrical cap 140 along at least a portion of the second axis 142 is selected from the group consisting of substantially oval, substantially hexagonal, substantially octagonal, and substantially polygonal.

In embodiments, the exterior cross-sectional shape of the cylindrical case 120 is elliptical along at least a portion of the first axis 122. In embodiments, the exterior cross-sectional shape of the cylindrical cap 140 is elliptical along at least a portion of the second axis 142. The exterior cross-sectional shape of the cylindrical case 100 may be elliptical for at least 50% of the length of the cylindrical case 100 along the first axis 122. The exterior cross-sectional shape of the cylindrical case 100 may be elliptical for at least 60%, at least 70%, at least 80%, or at least 90%, of the length of the cylindrical case 100 along the first axis 122. The exterior cross-sectional shape of the cylindrical cap 140 may be elliptical for at least 50% of the length of the cylindrical cap along the second axis 142. The exterior cross-sectional shape of the cylindrical cap 140 may be elliptical for at least 60%, at least 70%, at least 80%, or at least 90%, of the length of the cylindrical cap along the second axis 142. While an oblong cross-sectional shape is desirable for the reasons discussed above, an elliptical cross-sectional shape is even more preferable for comfort and security in view of the smooth, symmetrical feel when gripped resulting therefrom.

In embodiments, the elliptical exterior cross-sectional shape of the cylindrical case 120 has an average ratio of major axis length to minor axis length of 1.1:1 to 2.5:1 along the first axis 122. The elliptical exterior cross-sectional shape of the cylindrical case 120 preferably has an average ratio of major axis length to minor axis length of 1.1:1 to 2:1, more preferably 1.3:1 or about 1.3:1 along the first axis 122. In embodiments, the elliptical exterior cross-sectional shape of the cylindrical cap 140 has an average ratio of major axis length to minor axis length of 1.1:1 to 2.5:1 along the second axis 142. The elliptical exterior cross-sectional shape of the cylindrical cap 140 preferably has an average ratio of major axis length to minor axis length of 1.1:1 to 2:1, more preferably 1.3:1 or about 1.3:1 along the second axis 142.

While an oblong cross-sectional shape is desirable for the reasons discussed above, an elliptical cross-sectional shape is even more preferable for comfort and security in view of the smooth, symmetrical feel resulting therefrom. This is even more the case with major/minor axis ratios as defined above.

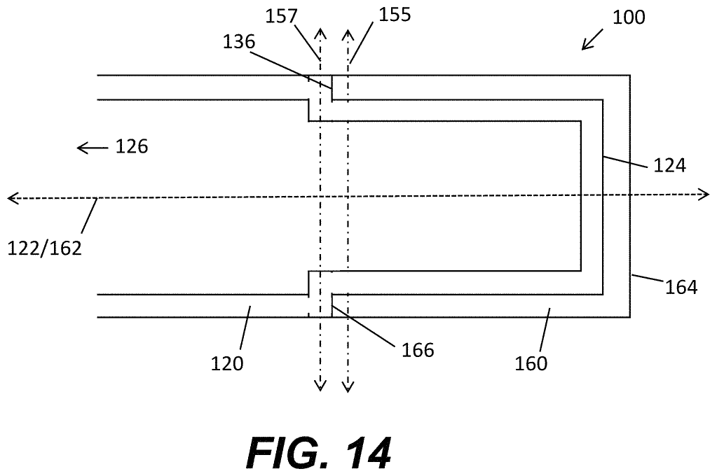

As noted above and shown in FIG. 1, the autoinjector housing 100 may further include a cylindrical overmolding 160. As shown in FIG. 13, the cylindrical overmolding 160 has a third axis 162 extending from a closed end 164 to an open end 166 of the cylindrical overmolding 160. The cylindrical overmolding 160 encloses an interior space accommodating at least a portion of the cylindrical case 120 such that the third axis coincides 162 with the first axis 122 (see FIG. 12). At least the closed handle end 124 of the cylindrical case 120 is covered by the cylindrical overmolding 160. The cylindrical overmolding 160 comprises a material having elasticity. Optionally, the material forming the cylindrical overmolding 160 has greater surface friction than one or more surfaces of the cylindrical case 120 and/or the cylindrical cap 140. The cylindrical overmolding 160 has a non-uniform radial thickness measured in a direction perpendicular to the third axis 122—of course, a cylindrical overmolding 160 having a uniform or substantially uniform radial thickness could also be employed.

As shown in FIG. 14, the exterior cross-sectional shape of the cylindrical case 120 is varied along the first axis 122 such that there is a step-up in cross-sectional area along the first axis 122 approaching the open injector end 126. The step-up in cross-sectional area of the cylindrical case 120 forms an overmolding flange 136, and the cylindrical overmolding 160 covers the cylindrical case 120 from the closed handle end 124 to the overmolding flange 136. The exterior cross-sectional shape of the cylindrical case 120 on an open-injector 126 side of the overmolding flange 136 and an exterior cross-sectional shape of the cylindrical overmolding 160 at the open end are substantially the same. The foregoing can be seen with reference to the first cross-section 155 and the second cross-section 157. Thus, there is a smooth transition in exterior cross-sectional shape from the cylindrical overmolding 160 to an adjacent portion of the cylindrical case 120 not covered by the cylindrical overmolding 160. A patient or caregiver desirably would not perceive a variation in the dimensions of the autoinjector housing 100 between the cylindrical overmolding 160 and the adjacent exposed portion of the cylindrical case 120.

Referring to FIG. 13, the cylindrical overmolding 160 comprises a plurality of openings 170 extending through, or at least partially through, the cylindrical overmolding 160 in a radial direction. Of course, a cylindrical overmolding 160 with no openings 170 could be used, and any combination of openings 170 that extend through or only partly through the cylindrical overmolding 160 could be used. When viewed from an exterior of the cylindrical overmolding 160, an exterior of the cylindrical case 120 is visible through each of the plurality of openings 170 that extends entirely through the cylindrical overmolding 160.

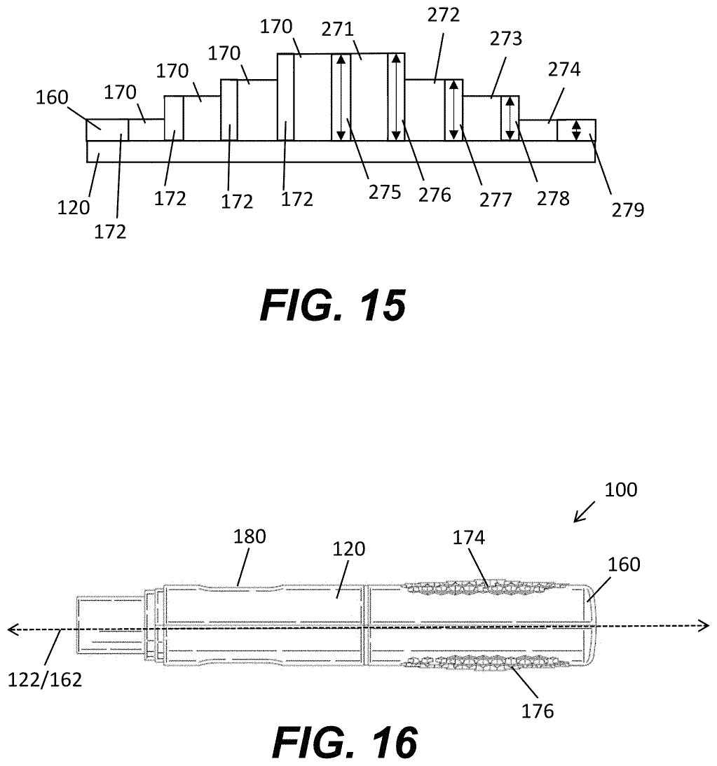

Referring to FIG. 15, the cylindrical overmolding 160 has a radial thickness (shown with vertical arrows) and openings 170. For the purpose explanation, certain specific openings are designated by the reference numbers 271, 272, 273, and 274, and certain specific sub-portions of the cylindrical overmolding 160 are designated by the reference numbers 275, 276, 277, 278, and 279 in FIG. 14. The thickness of the cylindrical overmolding 160 adjacent to and forming at least one of the openings 170 is greater than an average radial thickness of the cylindrical overmolding 160. This arrangement is shown, e.g., with respect to the opening 271 that is surrounded by the sub-portions 275 and 276 of the cylindrical overmolding 160. The sub-portions 275 and 276 have a greater radial thickness (shown with vertical arrow) than the radial thicknesses of the other sub-portions 277, 278, 279 of the cylindrical overmolding 160. In some embodiments, at least one opening 170 is bounded by at least two sub-portions of the cylindrical overmolding 160 having different radial thicknesses. This is the case with respect to the opening 272 surrounded by the sub-portion 276 and the sub-portion 277, the sub-portion 276 having a greater radial thickness than the sub-portion 277 (and with the openings 273, 274 surrounded by the sub-portions 277 and 278, and the sub-portions 278 and 279, respectively).

In embodiments, the radial thickness of the cylindrical overmolding 160 adjacent to and forming a first one of the plurality of openings 170 is greater than the radial thickness of the cylindrical overmolding 160 adjacent to and forming a second one of the plurality of openings. This is the case with respect to the opening 271 surrounded by sub-portions 275 and 276 and the opening 273 surrounded by the sub-portions 277 and 278, the opening 271 is surrounded by sub-portions of the cylindrical overmolding 160 having greater height than the sub-portions surrounding the opening 273. Of course, it also possible to provide a plurality of openings 170 in which the radial thickness the cylindrical overmolding 160 adjacent to and forming each of the plurality of openings 170 is substantially uniform. In such case or in other cases in which there is no gradient in opening 170 depth as shown in FIG. 15, distinct edges of the respective openings 170 (e.g., a vertex where the wall of an opening 170 meets an adjacent exterior surface of the cylindrical overmolding 160 that is perceivable to the touch) may be sufficient to increase traction and/or grippiness of the cylindrical overmolding 160 when handled by a patient or caregiver.

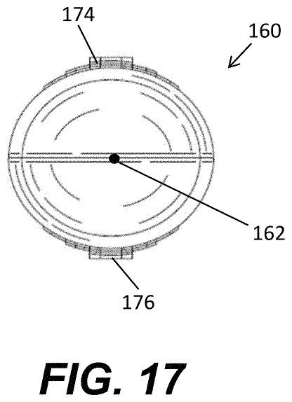

As shown in FIGS. 16 and 17, in embodiments, the plurality of openings 170 comprises a first grouping 174 of openings 170 arranged in a grid or matrix when viewed from the exterior of the cylindrical overmolding 160 and a second grouping 176 of openings 170 arranged in a grid or matrix when viewed from the exterior of the cylindrical overmolding 160. In embodiments, first grouping 174 and the second grouping 176 are arranged on opposite sides of the third axis 162 of the cylindrical overmolding 160 (which coincides with the first axis 122 of the cylindrical case 120).

As shown in FIG. 17, in embodiments, an exterior cross-sectional shape of the cylindrical overmolding 160 is oblong along an entirety of the third axis 162. In such embodiments, the first grouping 174 of openings 170 and the second grouping 176 of openings 170 are arranged on opposite sides of a longest dimension of cylindrical overmolding 160 in a direction perpendicular to the third axis 162 (i.e. the longest horizontal dimension in FIG. 17).

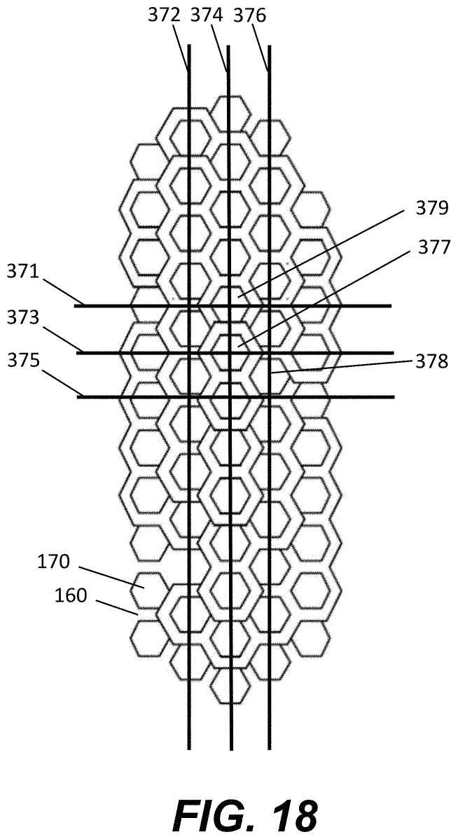

As shown in, e.g., FIG. 13, in embodiments, the plurality of openings 170 includes at least one grouping of openings 170 arranged in a grid or matrix when viewed from an exterior of the cylindrical overmolding 160. An exemplary grid or matrix formation is shown in FIG. 18 with vertical and horizontal lines added to show rows and columns of openings 170.

As shown in FIG. 18, in embodiments, at least one grouping of openings 170 includes openings arranged in a grid or matrix. In embodiments, the grid or matrix includes at least three adjacent rows 371, 373, 375 of openings 170 (shown with lines over the respective rows) extending along the cylindrical overmolding 160 in a circumferential direction (horizontal direction in FIG. 18; compare FIG. 13), including at least one central row 373 and at least two peripheral rows 371, 375. The grid or matrix includes at least three adjacent columns 372, 374, 376 of openings 170 (shown with lines over the respective columns) extending along the cylindrical overmolding 160 in an axial direction (vertical direction in FIG. 18; compare FIG. 13), including at least one central column 374 and at least two peripheral columns 372, 376. The radial thickness of at least a portion of the cylindrical overmolding 160 adjacent to and forming at least one of the openings 170 in the central row 373 is greater than the radial thickness of at least a portion of the cylindrical overmolding 160 adjacent to and forming at least one of the openings 170 in at least one of the peripheral rows 371, 375. At least a portion of the cylindrical overmolding 160 surrounding the opening 377 in row 373 may have a greater radial thickness than at least a portion of the cylindrical overmolding 160 surrounding opening 379 in adjacent row 371. The radial thickness of at least a portion of the cylindrical overmolding 160 adjacent to and forming at least one of the openings 170 in the central column 374 is greater than the radial thickness of at least a portion of the cylindrical overmolding 160 adjacent to and forming at least one of the openings 160 in at least one of the peripheral columns 372, 376. For example, a least a portion of the cylindrical overmolding 160 surrounding the opening 377 in column 374 has a greater radial thickness than at least a portion of the cylindrical overmolding 160 surrounding opening 378 in adjacent row 376.

In various exemplary embodiments, the plurality of openings 170 includes openings 170 that are polygonal in shape. The openings shown in FIGS. 13 and 18, e.g., are hexagonal in shape. However other polygons such as triangles, squares and other quadrilaterals, pentagons, octagon, dodecahedrons, or combinations thereof may be used. The shape of the openings is not particularly limited—circles, ovals, elipses, even symbols, could be used—and any combination of shapes may used to achieve a visual effect and/or improve grip performance.

In embodiments, the at least one grouping of openings 170 extends over at least 10% of an exterior surface area of the cylindrical overmolding 160. In embodiments, the at least one grouping of openings 170 extends over at least 20%, at least 30%, or at least 40% of an exterior surface area of the cylindrical overmolding 160. In embodiments, one or more groupings of openings 170 includes a plurality of hexagonal shaped openings 170 arranged in a honeycomb grid.

A length of the cylindrical overmolding 160 along the third axis 162 from the closed end 164 to the open end 166 may preferably be (i) greater than or equal to 55 mm and less than or equal to 85 mm, (ii) greater than or equal to 65 mm and less than or equal to 75 mm, (iii) any combination of greater than or equal to 55 mm, 60 mm, or 65 mm, and less than or equal to 75 mm, 80 mm, or 85 mm, or (iv) even more preferably 70 mm or about 70 mm.

An average major dimension of the exterior cross-sectional shape of the cylindrical overmolding 160 along the third axis 162 from the closed end 164 to the open end 166 may preferably be (i) greater than or equal to 20 mm and less than or equal to 40 mm, (ii) greater than or equal to 25 mm and less than or equal to 30 mm, (iii) any combination of greater than or equal to 20 mm, or 25 mm, and less than or equal to 30 mm, 35 mm, or 40 mm, or (iv) even more preferably 28 mm or about 28 mm.

An average minor dimension of the exterior cross-sectional shape of the cylindrical overmolding 160 along the first axis 122 from the closed handle end 124 to the open injector end 126 may preferably be (i) greater than or equal to 15 mm and less than or equal to 35 mm, (ii) greater than or equal to 20 mm and less than or equal to 25 mm, (iii) any combination of greater than or equal to 15 mm, or 20 mm, and less than or equal to 25 mm, 30 mm, or 35 mm, or (iv) even more preferably 23 mm or about 23 mm.

An average diameter of each opening 170 of the cylindrical overmolding 160 (measured as the diameter of a smallest circle enclosing the opening 170 when viewing the opening 170 from an exterior of the cylindrical overmolding 160) may preferably be (i) greater than or equal to 2 mm and less than or equal to 5 mm, (ii) greater than or equal to 2.5 mm and less than or equal to 3.5 mm, (iii) any combination of greater than or equal to 2 mm, 2.5 mm, and less than or equal to 3.5 mm, 4 mm, 4.5 mm, or 5 mm, or (iv) even more preferably 3 mm or about 3 mm.

An average radial thickness of the cylindrical overmolding 160 may preferably be (i) greater than or equal to 0.3 mm and less than or equal to 2.1 mm, (ii) greater than or equal to 0.3 mm and less than or equal to 0.9 mm, (iii) any combination of greater than or equal to 0.3 mm and less than or equal to 0.9 mm, 1.2 mm, 1.5 mm, 1.8 mm, or 2.1 mm, or (iv) even more preferably 0.6 mm or about 0.6 mm.

The shape and location of the cylindrical overmolding 160 and its openings 170 provide distinct advantages to the autoinjector housing 100 relative to known autoinjector housings. Particularly, the cylindrical overmolding 160 is provided over the closed handle end 124 of the cylindrical case 120. This arrangement is reminiscent of the grip of a golf club or tennis racket and, thus, signals to a patient or caregiver that the cylindrical overmolding 160 is the part of the autoinjector housing for grasping. This is even further reinforced by the openings 170 which are reminiscent of openings in the grip of a golf club or tennis racket. The cylindrical overmolding 160 follows the oblong cross-sectional shape of the cylindrical case 120 that it covers, thus conserving the advantages of such a cross-sectional shape discussed above. In addition, because portions of the cylindrical overmolding 160 surrounding the openings 170 may be of variable radial thickness and, in embodiments, the portions of greater radial thickness maybe located centrally in a grouping of openings 170, a patient or caregiver can more easily identify where to grip the autoinjector housing 100 so as to fit into the palm of the hand, making the grip more secure. Further, because plural groupings of openings 170 may be provided on opposite sides of the cylindrical overmolding 160 and preferably on opposite sides of a longest dimension of the cylindrical overmolding 160 in a direction perpendicular to the third axis 162, the visual cues of the cylindrical overmolding 160 which suggest that it should be gripped (e.g. the openings 170) can be seen when the autoinjector 100 is laid lengthwise on the flat surface of a table.

In embodiments, the cylindrical overmolding 160 is formed by molding a polymeric material. In embodiments, the cylindrical overmolding 160 is formed by molding a thermoplastic elastomer. In embodiments, the cylindrical overmolding 160 is formed of a polymeric material that, after molding and post-molding processing, has good slip resistance and an elastic touch. In embodiments, the cylindrical overmolding 160 is formed of a polymeric material that, after molding and processing, is opaque and colored. In embodiments, the polymeric material is gray. In embodiments, the cylindrical overmolding 160 is formed of a polystyrene thermoplastic elastomer.

In embodiments, the cylindrical cap 140 and the cylindrical overmolding 160 differ in at least one surface characteristic. The cylindrical cap 140 and the cylindrical overmolding 160 may differ in color. Alternatively, the cylindrical cap 140 and the cylindrical overmolding 160 differ in texture. In further embodiments, the cylindrical case 120, the cylindrical cap 140, and the cylindrical overmolding 160 may differ in at least one surface characteristic. The cylindrical case 120, the cylindrical cap 140, and the cylindrical overmolding 160 may differ in color. The cylindrical case 120, the cylindrical cap 140, and the cylindrical overmolding 160 may differ in texture.

Surface characteristics may describe any aspect of the materials forming surfaces of the autoinjector housing 100 that are perceivable by a patient or caregiver using the autoinjector housing 100 and, particularly visual or textural characteristics. Visual surface characteristics may include color or pattern. Color may result from the color of polymeric materials (natural or imparted by colorants) used to form components of the autoinjector housing 100 and/or color added thereafter by dyeing, painting, printing, etc. Pattern may result from non-uniform coloration of polymeric materials used to form components of the autoinjector housing 100 and/or non-uniform color added thereafter by painting, printing, etc. Pattern may particular include printed subject matter provided on surfaces of the autoinjector housing 100, including, e.g., instructions for use (written instructions and/or illustrations) and branding. Texture may result from intrinsic properties of polymeric materials used to form components of the autoinjector housing 100 and/or texture added by virtue of molding and/or post-molding processing of a component of the autoinjector housing 100. Examples of intrinsic properties of polymeric materials include hardness, elasticity, tackiness, grippiness, smoothness, silkiness, etc. Examples texture resulting from molding can include any surface texture imparted by an interior of a mold, such as a pebbling. Examples of texture resulting from post-molding processing including any texture imparted by buffing, polishing, abrading, stamping, etc.

Differences in surface characteristics of the autoinjector housing 100 as between the cylindrical case 120, the cylindrical cap 140, the cylindrical overmolding 160, and the retractable needle shield 300 (part of the autoinjector mechanism) are important in influencing how a patient or caregiver uses an autoinjector. In embodiments, each of these features differs in at least one surface characteristic. In embodiments, the cylindrical case 120, the cylindrical cap 140, the cylindrical overmolding 160, and the retractable needle shield 300 each have a different color. Preferably, the retractable needle shield 300 is of a bright, bold color (e.g., yellow, orange, red), e.g., to warn that the feature is used for injection and care should be taken. Preferably, the cylindrical cap 140 is transparent or translucent, e.g., so that the retractable needle shield can be seen when the cylindrical cap 140 is attached to the cylindrical case 120. Preferably, the cylindrical case 120 is provided in a color permitting clear contrasting printing thereon. Preferably, the cylindrical case 120 and the cylindrical overmolding 160 are of contrasting color, e.g., so that a patient or caregiver will discern that the autoinjector housing should be gripped at the cylindrical overmolding 160. Further, this contrast will permit the color of the cylindrical case 120 to be seen through openings 170 in the cylindrical overmolding 160 that extend therethrough, e.g., further reinforcing that the cylindrical overmolding 160 is for gripping. Differences in texture between the cylindrical case 120, the cylindrical cap 140, the cylindrical overmolding 160 can also improve the patient or caregiver experience. Particularly, it is desirable that cylindrical case 120 be smooth so as not to obstruct or obscure printing. It is desirable that the cylindrical cap 140 has some raised texture of roughening for grasping during removal of the cylindrical cap 140. Further, it desirable that cylindrical overmolding 160 have a rubbery, grippy, or tacky feel (together with the texture provided by the openings 170) to indicate that the cylindrical overmolding 160 should be gripped and to increase the effectiveness of the grip.

By incorporating appropriate contrasting materials in an autoinjector housing 100 as described herein, it is possible to facilitate correct use of an autoinjector in a two-step injection process (remove the cap and press into injection site). Particularly, the contrasting materials make the directionality of the autoinjector housing 100 (i.e., the handle end and the injector end) and the function of its respective parts more intuitive and effective and, thus, less prone to injury and errors in injection.

Differences in surface characteristics of the autoinjector housing 100 as between the cylindrical case 120, the cylindrical cap 140, the cylindrical overmolding 160, and the retractable needle shield 300 (part of the autoinjector mechanism) are also important to allow differentiation between autoinjector housings 100 of otherwise like construction (i.e., for reasons other than patient/caregiver safety and ease of use). For example, selecting different colors for the retractable needle shield 300 of otherwise like autoinjectors, could permit differentiation between branding, medicaments, dosages, etc., in the respective autoinjectors.

The present disclosure is further directed to employing the autoinjector housing 100 as described herein in an autoinjector. In embodiments, such an autoinjector includes an autoinjector housing 100 as described herein and an autoinjector mechanism. The autoinjector housing 100 described herein, and thus an autoinjector incorporating such housing, is intended to be actuated by pressing the entire autoinjector into the injection site (e.g., causing the retractable needle shield 300 to retract during pressing). Thus, the autoinjector does not include a separate button or actuator that must be pressed by a patient or caregiver after the autoinjector is positioned at the injection site—the autoinjector is “button-less.” Thus, the after removing the cylindrical cap 140, the autoinjector can be operated one-handed. In embodiments, the autoinjector mechanism includes a syringe filled with a single dose of a medicament and a retractable needle shield (e.g., retractable needle shield 300 in FIG. 4) that surrounds a needle of the syringe. The retractable needle shield 300 of the autoinjector is provided in the opening 132 in the open injector end 126 of the cylindrical case 120.

Any known or later developed autoinjector mechanism that can be provided an autoinjector housing as described herein and perform “one-handed,” “button-less” injection as described above may be used. In embodiments, the autoinjector mechanism is an autoinjector mechanism as used in the YpsoMate 2-step injector, developed by Ypsomed AG of Burgdorf, Switzerland. In further embodiments, the autoinjector mechanism is an autoinjector mechanism as described in US 2016/0008541 A1. Moreover, any conventional molding and assembly techniques may be employed to manufacture components of the autoinjector housings described herein, the autoinjector housings described herein, and the autoinjectors described herein.

In embodiments, autoinjectors according to the present disclosure may be provided with a syringe filled with a single dose of adalimumab. The syringe may alternatively be filled with another tumor necrosis factor (TNF) blocker.

Adalimumab is a tumor necrosis factor (TNF) blocker. Other TNF blockers may alternatively be used. Adalimumab is a recombinant human IgG1 monoclonal antibody created, e.g., using phage display technology, resulting in an antibody with human derived heavy and light chain variable regions and human IgG1 kappa constant regions. Adalimumab is produced, e.g., by recombinant DNA technology in a mammalian cell (Chinese Hamster Ovary (CHO)) expression system and may be purified by a process that includes specific viral inactivation and removal steps. Adalimumab consists of 1330 amino acids and has a molecular weight of approximately 148 kilodaltons. In embodiments, a solution of adalimumab is clear and colorless, with a pH of 5.0 to 5.6. Adalimumab, as used in autoinjectors described herein, may be a sterile, preservative-free solution for subcutaneous administration. Single doses may be provided in, e.g., a 1 mL prefilled glass syringe (which are adaptable for use in and may, in turn, be provided in an autoinjector).

In embodiments, the single dose of adalimumab is 40 mg of adalimumab at a concentration of 100 mg/ml, i.e., 40 mg/0.4 mL. A 40 mg/0.4 mL prefilled syringe adaptable for use in an autoinjector delivers 0.4 mL (40 mg) of drug product. In embodiments, a single low dose of adalimumab contains adalimumab (40 mg), mannitol (16.8 mg), polysorbate 80 (0.4 mg), and Water for Injection, USP. In further embodiments, 40 mg/0.4 mL prefilled syringe adaptable for use in an autoinjector delivers adalimumab (40 mg), sodium chloride (0.23 mg), sucrose (34.9 mg), polysorbate 80 (0.80 mg), and Water for Injection, USP. Sodium hydroxide and/or hydrochloric acid may be added as necessary to adjust pH.

In embodiments, the single 40 mg/0.4 mL dose of adalimumab includes: (i) 100 mg/ml adalimumab, (ii) about 250 mM trehalose or sucrose, (iii) 0.1% polysorbate 20, and (iv) 20 mM acetate buffer.

In embodiments, the single dose of adalimumab is 80 mg of adalimumab at a concentration of 100 mg/ml, i.e., 80 mg/0.8 mL. A 80 mg/0.8 mL prefilled syringe adaptable for use in an autoinjector delivers 0.8 mL (80 mg) of drug product. In embodiments, a single high dose of adalimumab contains adalimumab (80 mg), mannitol (33.6 mg), polysorbate 80 (0.8 mg), and Water for Injection, USP. In further embodiments, 40 mg/0.4 mL prefilled syringe adaptable for use in an autoinjector delivers adalimumab (80 mg), sodium chloride (0.46 mg), sucrose (69.8 mg), polysorbate 80 (1.6 mg), and Water for Injection, USP. Sodium hydroxide and/or hydrochloric acid may be added as necessary to adjust pH.

In embodiments, the syringe comprises an adalimumab formulation as described in WO 2020/187760 A1 is used. In embodiments, the formulation includes: (i) 90-120 mg/ml adalimumab, (ii) about 200 to 275 mM trehalose or sucrose, (iii) 0.05-0.15% nonionic surfactant, and (iv) 25 mM or less acetate or succinate buffer

In embodiments, the single dose of adalimumab is 40 mg of adalimumab at a concentration of 50 mg/ml, i.e., 40 mg/0.8 mL. A 40 mg/0.8 mL prefilled syringe adaptable for use in an autoinjector delivers 0.8 mL (40 mg) of drug product. In embodiments, a single dose of adlimumab includes adalimumab (40 mg), citric acid monohydrate (1.04 mg), dibasic sodium phosphate dihydrate (1.22 mg), mannitol (9.6 mg), monobasic sodium phosphate dihydrate (0.69 mg), polysorbate 80 (0.8 mg), sodium chloride (4.93 mg), sodium citrate (0.24 mg) and Water for Injection, USP. Sodium hydroxide may be added as necessary to adjust pH.

In alternative embodiments, the single dose of adalimumab may be 20 mg/0.2 mL, 20 mg/0.4 mL, 10 mg/0.1 mL, or 10 mg/0.2 mL.

The present disclosure is further directed to methods of treating various diseases or disorders, comprising administering a single dose of adalimumab to a patient in need of such treatment with an autoinjector as described herein. Exemplary diseases or disorders include one or more of rheumatoid arthritis, juvenile idiopathic arthritis, psoriatic arthritis, ankylosing spondylitis, Crohn's disease, ulcerative colitis, plaque psoriasis, hidradenitis suppurativa, and uveitis. Other tumor necrosis factor (TNF) blockers may be used to treat other autoimmune and immune-mediated disorders. In embodiments, methods include self-administering by the patient. In embodiments, a single dose is administered weekly or every other week.

In alternative embodiments, autoinjectors according to the present disclosure may be provided with a syringe filled with a single dose of an active ingredient selected form the group consisting of golimumab, infliximab, certolizumab pegol, and etanercept.

In embodiments, methods may include administering a single dose of golimumab to a patient in need of treatment of rheumatoid arthritis, psoriatic arthritis, ankylosing spondylitis, or ulcerative colitis. In embodiments, methods include self-administering by the patient. In embodiments, a single dose is administered every other week or monthly.

Golimumab is a human IgG1κ monoclonal antibody specific for human tumor necrosis factor alpha (TNFα) that exhibits multiple glycoforms with molecular masses of approximately 150 to 151 kilodaltons. In embodiments, golimumab may be created using genetically engineered mice immunized with human TNF, resulting in an antibody with human-derived antibody variable and constant regions. In embodiments, golimumab is produced by a recombinant cell line cultured by continuous perfusion and is purified by a series of steps that includes measures to inactivate and remove viruses. Golimumab may be a preservative-free, sterile, clear to slightly opalescent, colorless to light yellow solution of the golimumab antibody supplied in a single-dose prefilled syringe adaptable for use in an autoinjector. The solution may have a pH of approximately 5.5.

In embodiments, a single dose of golimumab may be in the form of a 50 mg/0.5 mL single-dose prefilled syringe (adaptable for use in and which may, in turn, be provided in an autoinjector). Each 0.5 mL prefilled syringe adaptable for use in an autoinjector may contain 50 mg golimumab, L-histidine and L-histidine monohydrochloride monohydrate (0.44 mg), polysorbate 80 (0.08 mg), sorbitol (20.5 mg) and Water for Injection. In embodiments, each 0.5 mL prefilled syringe adaptable for use in an autoinjector may contain 50 mg golimumab, L-histidine and L-histidine monohydrochloride monohydrate (0.44 mg), poloxamer 188 (0.08 mg), sorbitol (20.5 mg) and Water for Injection.

In embodiments, a single dose of golimumab may be in the form of a 100 mg/0.5 mL single-dose prefilled syringe (adaptable for use in and which may, in turn, be provided in an autoinjector). Each 1 mL prefilled syringe adaptable for use in an autoinjector may contain 100 mg golimumab, L-histidine and L-histidine monohydrochloride monohydrate (0.87 mg), polysorbate 80 (0.15 mg), sorbitol (41.0 mg) and Water for Injection. In embodiments, each 1 mL prefilled syringe adaptable for use in an autoinjector may contain 100 mg golimumab, L-histidine and L-histidine monohydrochloride monohydrate (0.87 mg), poloxamer 188 (0.15 mg), sorbitol (41.0 mg) and Water for Injection.

In embodiments, methods may include administering a single dose of infliximab to a patient in need of treatment of Crohn's disease, ulcerative colitis, rheumatoid arthritis, ankylosing spondylitis, psoriatic arthritis, or plaque psoriasis. In embodiments, methods include self-administering by the patient. In embodiments, a single dose is administered every other week, every six weeks, or every eight weeks.

Infliximab, a TNF blocker, is a chimeric IgG1K monoclonal antibody (composed of human constant and murine variable regions). It has a molecular weight of approximately 149.1 kilodaltons. Infliximab may be produced by a recombinant murine myeloma cell line, SP2/0. Infliximab may be supplied as a sterile, preservative-free, white, lyophilized powder for intravenous infusion after reconstitution and dilution. In embodiments, infliximab is reconstituted with 10 mL of Sterile Water for Injection, USP, to have a final concentration of 10 mg/mL and a resulting pH of approximately 7.2.

In embodiments, a single dose of infliximab may be in the form of a 10 mg/mL single-dose prefilled syringe (adaptable for use in and which may, in turn, be provided in an autoinjector). Prior to reconstitution a vial may include 100 mg infliximab, dibasic sodium phosphate, dihydrate (6.1 mg), monobasic sodium phosphate, monohydrate (2.2 mg), polysorbate 80 (0.5 mg), and sucrose (500 mg).

In embodiments, methods may include administering a single dose of certolizumab pegol to a patient in need of treatment of Crohn's disease, rheumatoid arthritis, psoriatic arthritis, ankylosing spondylitis, spondyloarthritis, or plaque psoriasis. In embodiments, methods include self-administering by the patient. In embodiments, a single dose is administered every other week, every six weeks, or every eight weeks.

Certolizumab pegol is a TNF blocker. Certolizumab pegol is a recombinant, humanized antibody Fab′ fragment, with specificity for human tumor necrosis factor alpha (TNFα), conjugated to an approximately 40 kilodaltons polyethylene glycol (PEG2MAL40K). The Fab′ fragment may be manufactured in E. coli and may be subsequently subjected to purification and conjugation to PEG2MAL40K, to generate certolizumab pegol. The Fab′ fragment is composed of a light chain with 214 amino acids and a heavy chain with 229 amino acids. The molecular weight of certolizumab pegol is approximately 91 kilodaltons. Certolizumab pegol may be supplied as a sterile, clear to opalescent, colorless to pale yellow solution that may contain particulates in a single-dose prefilled syringe for subcutaneous use in an autoinjector.

In embodiments, a single dose of certolizumab pegol may be in the form of a 200 mg/mL single-dose prefilled syringe adaptable for use in an autoinjector (which may, in turn, be provided in an autoinjector). Each 1 mL prefilled syringe adaptable for use in an autoinjector may contain 200 mg certolizumab pegol, sodium acetate (1.36 mg), sodium chloride (7.31 mg), and Water for Injection, USP.

In embodiments, methods may include administering a single dose of etanercept to a patient in need of treatment of rheumatoid arthritis, juvenile idiopathic arthritis, psoriatic arthritis, ankylosing spondylitis, or plaque psoriasis. In embodiments, methods include self-administering by the patient. In embodiments, a single dose is administered once a week or twice a week.

Etanercept, a TNF blocker, is a dimeric fusion protein consisting of the extracellular ligand-binding portion of the human 75 kilodalton (p75) tumor necrosis factor receptor (TNFR) linked to the Fc portion of human IgG1. The Fc component of etanercept contains the C_{H}2 domain, the C_{H}3 domain and hinge region, but not the C_{H}1 domain of IgG1. Etanercept may be produced by recombinant DNA technology in a Chinese hamster ovary (CHO) mammalian cell expression system. It consists of 934 amino acids and has an apparent molecular weight of approximately 150 kilodaltons. Etanercept may be supplied as a clear and colorless, sterile, preservative-free solution having a pH of 6.3±0.2.

In embodiments, a single dose of etanercept may be in the form of a 50 mg/mL single-dose prefilled syringe (adaptable for use and which may, in turn, be provided in an autoinjector). Each 1 mL prefilled syringe adaptable for use in an autoinjector may contain 50 mg etanercept, 25 mM L-arginine hydrochloride, 120 mM sodium chloride, and 1% sucrose.

In embodiments, a single dose of etanercept may be in the form of a 25 mg/mL single-dose prefilled syringe (adaptable for use and which may, in turn, be provided in an autoinjector). Each 1 mL prefilled syringe adaptable for use in an autoinjector may contain 25 mg etanercept, 25 mM L-arginine hydrochloride, 120 mM sodium chloride, and 1% sucrose.

The present disclosure is further directed to methods of injecting a medicament using an autoinjector as described herein. In embodiments, methods include removing the cylindrical cap 140 from the cylindrical case 120, if attached, grasping the autoinjector, positioning the autoinjector so the retractable needle shield 300 is in contact with an injection site on a patient's skin and the first axis 122 of cylindrical case 120 is substantially perpendicular to the injection site, pressing the autoinjector into the skin in a direction substantially perpendicular to the injection site to cause (i) the needle to insert into the injection site, (ii) the retractable needle shield 300 to retract into the cylindrical case 120, and (iii) the single dose of the medicament to be injected into the injection site, and pulling the autoinjector away from the injection site resulting in return of the retractable needle shield 300 to an unretracted state when administration of the single dose of the medicament is complete. Grasping the autoinjector may include grasping at any location along the length of the autoinjector. In embodiments, the autoinjector may be grasped at or about the closed handle end 124 of the cylindrical case 120. In alternative embodiments, the autoinjector may be grasped at a central location along its length. In embodiments, the method may further include waiting or allowing the contents of the autoinjector (syringe) to enter the injection site after pressing the autoinjector into the skin and before pulling the autoinjector away from the injection site. In embodiments, upon return of the retractable needle shield 300 to the unretracted state, the retractable needle shield 300 is locked in the unretracted state so that the patient or caregiver is prevented from interacting with the needle therein (thus reducing the likelihood of blood borne illness, needle stick injury, etc.).

In embodiments, after removing the cylindrical cap 140, the method is performed one-handed. In embodiments, the method is performed without pressing an actuator or button. In embodiments, grasping the autoinjector comprises grasping the cylindrical overmolding 160.

In embodiments, methods of injecting a medicament using an autoinjector as described herein may include removing the autoinjector from a refrigerator and leaving at room temperature for 15 to 30 minutes before injecting. In embodiments, the foregoing steps may be performed without removing the cap (e.g., cylindrical cap 140) from the autoinjector housing (e.g., cylindrical case 120). In embodiments, the foregoing steps may be performed without warming the autoinjector, e.g., in a microwave or in hot water. In embodiments, the foregoing steps are not performed if the contents of the autoinjector have been frozen, even if the contents have subsequently been thawed.

In embodiments, methods of injecting a medicament using an autoinjector as described herein may include checking the expiration date, gathering ancillary supplies, and washing hands. In embodiments, the foregoing steps may include checking the expiration date on the autoinjector or packaging therefor. In embodiments, the foregoing steps may include discarding the autoinjector if the expiration date has passed. In embodiments, the foregoing steps may include placing the autoinjector, an alcohol swab, a cotton ball or gauze pad, and/or a puncture-resistant sharps disposal container on a clean, flat surface. In embodiments, the foregoing steps may include washing and drying a patient or caregiver's hands.

In embodiments, methods of injecting a medicament using an autoinjector as described herein may include choosing and cleaning an injection site. In embodiments, the foregoing steps may include choosing an injection site on the front of a patient's thigh, on the patient's abdomen (belly) at least 2 inches from the patient's navel (belly button), and at least one inch from the patient's last injection site (if any). In embodiments, the foregoing steps may include wiping the injection site in a circular motion with an alcohol swab. In embodiments, methods do not include injecting through clothes. In embodiments, the foregoing steps do not include injecting into skin that is sore, bruised, red, hard, scarred, has stretch marks, or areas with psoriasis plaques.

In embodiments, methods of injecting a medicament using an autoinjector as described herein may include checking medicine in an inspection window (e.g., window opening 180). In embodiments, the foregoing step may include holding the autoinjector with a grip area (e.g., openings 170 of cylindrical overmolding 160) facing up. In embodiments, the foregoing step may include checking the inspection window. In embodiments, the foregoing step may include confirming that a liquid visible in the inspection window is clear and colorless, although bubbles may be present. In embodiments, the foregoing step includes discarding the autoinjector if the liquid visible in the inspection window is cloudy or has particles. In embodiments, the foregoing step includes discarding the autoinjector if the autoinjector has been dropped or crushed.

In embodiments, methods of injecting a medicament using an autoinjector as described herein may include removing the cap (e.g., cylindrical cap 140). In embodiments, the foregoing step may include pulling the cap straight off. In embodiments, when the cap is removed, a few drops of liquid may come out of a needle of the autoinjector. In embodiments, the foregoing step may include throwing the cap away. In embodiments, the foregoing step does not include replacing the cap on the pen to avoiding possible damage to the needle. In embodiments, the foregoing step includes pointing a retractable needle shield (e.g., retractable needle shield 300) of the autoinjector at the injection site.

In embodiments, methods of injecting a medicament using an autoinjector as described herein may include pinching skin at the injection site and positioning the autoinjector over the injection site. In embodiments, the foregoing steps may include squeezing the skin at the injection site to make a raised area and holding the pinched skin firmly so as to temporarily increase the depth of the injection area to ensure that delivery is subcutaneous, and not intramuscular. In embodiments, the foregoing steps may include placing the retractable needle sleeve straight (90° angle) against the injection site. In embodiments, the foregoing steps may include holding the autoinjector so that you can see the inspection window.

In embodiments, methods of injecting a medicament using an autoinjector as described herein may include giving an injection. In embodiments, the foregoing step may include pushing and continuing to push the autoinjector down against the injection site. In embodiments, pushing the autoinjector down against the injection site will cause the autoinjector to make a clicking noise when the injection begins. In embodiments, the foregoing step includes pushing the autoinjector down against the injection until the retractable needle shield stops moving and the autoinjector makes a second clicking noise to indicate the injection is complete. The time from the first clicking noise to the second clicking noise may preferably be (i) greater than or equal to 3 seconds and less than or equal to 15 seconds, (ii) greater than or equal to 8 seconds and less than or equal to 12 seconds, (iii) any combination of greater than or equal 3, 4, 5, 6, 7, or 9 seconds, and less than or equal to 12, 13, 14, or 15 seconds, or (iv) even more preferably 10 seconds or about 10 seconds. In embodiments, the foregoing step includes not lifting up, or letting go of pressure from the injection site, until it is confirmed that the injection is complete.

In embodiments, methods of injecting a medicament using an autoinjector as described herein may include removing the autoinjector from the skin and performing post-injection care. In embodiments, the foregoing steps may include, when the injection is completed, slowly pulling the autoinjector from the skin. In embodiments, the retractable needle shield will return to cover the needle as the autoinjector is pulled from the skin. In embodiments, the foregoing steps may include contacting a healthcare provider if there are more than a few drops of liquid on the injection site after the autoinjector is pulled from the skin. In embodiments, the foregoing steps include, after completing the injection, placing a cotton ball or gauze pad on the skin of the injection site, optionally followed by securing the cotton or gauze with non-toxic, medically-approved adhesive. In embodiments, the foregoing steps do not include rubbing the injection site. Slight bleeding at the injection site is normal during the foregoing steps.

The foregoing methods employing autoinjectors as described herein provide distinct advantages over prior methods and autoinjectors in intuitive operation, safe operation, and effective administration of a dose of a liquid medicament during the course of self-injection by a patient or administration by a caregiver. The active steps may include removing a single cap and pressing into the injection site (compared with devices having two caps and/or a separate button to effect injection after the autoinjector is placed on the injection site). The methods and devices described herein are simpler, allowing a patient or caregiver to more intuitively perceive which end of the autoinjector includes a needle. Exemplary features of autoinjector housings described above are particularly advantageous in the foregoing methods. The absence of a visible needle may reduce the risk of inadvertent needle sticks. Inclusion of a cap having an edge facilitates removal. Inclusion of a rubberized and textured grip together with an oblong cross-sectional shape and shorter length makes it easier to grip and hold the autoinjector in place during injection. Further, it is particularly advantageous to use an autoinjector that indicates injection duration with audible cues, such as clicks, at start and stop (in addition to the visual stop of the retractable needle shield becoming fully retracted), does not include a separate button or actuator, and completes injection in 10 seconds instead of 15 or more seconds. This unique function, which is particularly desirable for ensuring effective injection, is possible when employing an autoinjector mechanism as in the YpsoMate autoinjector referenced above. Also, injection integrity is improved because the act of injection involves only two principal elements: (i) holding the autoinjector in the palm, and (ii) continuously applying pressure to the injection site. These aspects result in intuitive operation, safe operation, and effective administration of a dose of a liquid medicament.

Any one or more of the foregoing method steps may be performed by a patient himself or herself, by a caregiver (healthcare professional, relative, etc.), or some combination thereof.

In embodiments, autoinjectors according to the present disclosure may be provided in a kit with instructions. Instructions may direct a patient or caregiver to perform any one or more of the method steps outlined above. Full or partial instructions may be provided on one or more sheets or in a booklet provided in the kit. Full or partial instructions may be provided on kit packaging. Full or partial instructions may be provided on autoinjector housings as described herein. The kit may further include ancillary components, such as a cotton ball, gauze pad, alcohol swab, and/or puncture-resistant sharps disposal container.

In various exemplary embodiments, the present disclosure relates the following items and all combinations thereof:

1. An autoinjector housing, comprising:

a cylindrical case enclosing an interior space capable of accommodating an autoinjector mechanism; and

a cylindrical cap removably attachable to the cylindrical case;

wherein:

the cylindrical case has a first axis extending from a closed handle end to an open injector end of the cylindrical case;

an exterior cross-sectional shape of the cylindrical case is varied along the first axis such that there is a step-down in cross-sectional area along the first axis approaching the open injector end;

the exterior cross-sectional shape of the cylindrical case is oblong along at least a portion of the first axis;

the open injector end of the cylindrical case comprises an opening capable of accommodating a retractable needle shield of the autoinjector mechanism;

the cylindrical cap has a second axis extending from an open end to a closed end of the cylindrical cap;

an exterior cross-sectional shape of the cylindrical cap is varied along the second axis such that there is a gradual decrease in cross-sectional area from the open end toward the closed end and a step-up in cross-sectional area approaching the closed end;

the exterior cross-sectional shape of the cylindrical cap is oblong along at least a portion of the second axis;

the cylindrical cap is removably attached to the cylindrical case by fitting the open end of the cylindrical cap over the open injector end of the cylindrical case such that the first axis and second axis are substantially aligned; and

the step-down in cross-sectional area of the cylindrical case forms a cap flange, and attachment of the cylindrical cap is completed when the open end of the cylindrical cap is seated on the cap flange.

2. The autoinjector housing of item 1 or any item depending therefrom, wherein:

the exterior cross-sectional shape of the cylindrical case is elliptical along at least a portion of the first axis; and

the exterior cross-sectional shape of the cylindrical cap is elliptical along at least a portion of the second axis.

3. The autoinjector housing of item 2 or any item depending therefrom, wherein:

the exterior cross-sectional shape of the cylindrical case is elliptical for at least 50% of the length of the cylindrical case along the first axis; and

the exterior cross-sectional shape of the cylindrical cap is elliptical for at least 50% of the length of the cylindrical cap along the second axis.

4. The autoinjector housing of item 2 or any item depending therefrom, wherein:

the elliptical exterior cross-sectional shape of the cylindrical case has an average ratio of major axis length to minor axis length of 1.1:1 to 2.5:1 along the first axis; and

the elliptical exterior cross-sectional shape of the cylindrical cap has a ratio of major axis length to minor axis length of 1.1:1 to 2.5:1 along the second axis.

5. The autoinjector housing of item 1 or any item depending therefrom, wherein:

the oblong exterior cross-sectional shape of the cylindrical case along at least a portion of the first axis is selected from the group consisting of substantially oval, substantially hexagonal, and substantially octagonal; and

the oblong exterior cross-sectional shape of the cylindrical cap along at least a portion of the second axis is selected from the group consisting of substantially oval, substantially hexagonal, and substantially octagonal.

6. The autoinjector housing of item 1 or any item depending therefrom, further comprising a cylindrical overmolding having a third axis extending from a closed end to an open end of the cylindrical overmolding; wherein:

the cylindrical overmolding encloses an interior space accommodating at least a portion of the cylindrical case such that the third axis coincides with the first axis;

at least the closed handle end of the cylindrical case is covered by the cylindrical overmolding; and

the cylindrical overmolding comprises a material having elasticity.

7. The autoinjector housing of item 6 or any item depending therefrom, wherein the cylindrical overmolding covers at least one third of a length of the cylindrical case along the first axis extending from the closed handle end.

8. The autoinjector housing of item 6 or any item depending therefrom, wherein the cylindrical cap and the cylindrical overmolding differ in at least one surface characteristic.

9. The autoinjector housing of item 8 or any item depending therefrom, wherein the cylindrical cap and the cylindrical overmolding differ in color.

10. The autoinjector housing of item 8 or any item depending therefrom, wherein the cylindrical cap and the cylindrical overmolding differ in texture.

11. The autoinjector housing of item 6 or any item depending therefrom, wherein the cylindrical case, the cylindrical cap, and the cylindrical overmolding differ in at least one surface characteristic.

12. The autoinjector housing of item 11 or any item depending therefrom, wherein the cylindrical case, the cylindrical cap, and the cylindrical overmolding differ in color.

13. The autoinjector housing of item 11 or any item depending therefrom, wherein the cylindrical case, the cylindrical cap, and the cylindrical overmolding differ in texture.

14. The autoinjector housing of item 1 or any item depending therefrom, wherein:

the cylindrical case comprises at least one window opening extending through the cylindrical case in a radial direction;

when the autoinjector mechanism is accommodated in the interior space and comprises a syringe, contents of the syringe can be seen through the window opening; and

the window opening has an oblong shape when viewed from an exterior of the cylindrical case with a major axis of the oblong shape being parallel to the first axis of the cylindrical case.

15. The autoinjector housing of item 1 or any item depending therefrom, wherein the cylindrical cap is the only cap of the autoinjector housing.

16. The autoinjector housing of item 1 or any item depending therefrom, wherein the autoinjector housing does not comprise an opening for accommodating a button or actuator.

17. The autoinjector housing of item 1 or any item depending therefrom, wherein the cylindrical cap comprises a transparent or translucent material.

18. The autoinjector housing of item 1 or any item depending therefrom, wherein:

the cylindrical cap comprises a first section, a second section, a third section, and a fourth section, in order, along the second axis from the open end to the closed end;

an exterior cross-sectional area in the first section is substantially constant;

the gradual decrease in cross-sectional area along the second axis from the open end toward the closed end of the cylindrical cap occurs in the second section;

the step-up in cross-sectional area along the second axis approaching the closed end of the cylindrical cap occurs in the third section; and

an exterior cross-sectional area of the cylindrical cap along the second axis is greater in the fourth section than at any point along the second axis in the first section, second section or third section.

19. The autoinjector housing of item 18 or any item depending therefrom, wherein:

the exterior cross-sectional shape of the cylindrical cap along the second axis in the second section is oblong; and

a major axis of the oblong exterior cross-sectional shape of the cylindrical cap decreases along the second axis in the second section.

20. The autoinjector housing of item 18 or any item depending therefrom, wherein the third section of the cylindrical cap comprises less than one tenth of a length of the cylindrical cap along the second axis.

21. The autoinjector housing of item 1 or any item depending therefrom, wherein:

the closed end of the cylindrical cap comprises a standing surface substantially perpendicular to the second axis; and

when the cylindrical cap is attached to the cylindrical case, the autoinjector housing can be stably stood on a flat surface by placing the standing surface in contact with the flat surface.

22. The autoinjector housing of item 19 or any item depending therefrom, wherein:

the standing surface of the closed end of the cylindrical cap is provided at a periphery of the closed end and surrounds an interior surface that is recessed with respect to the standing surface.

23. The autoinjector housing of item 11 or any item depending therefrom, wherein the interior surface comprises more than 75 percent of a surface area of the closed end of the cylindrical cap.

24. The autoinjector housing of item 1 or any item depending therefrom, wherein:

the cylindrical cap comprises an interior space that accommodates the open injector end of the cylindrical case when the cylindrical cap is attached to the cylindrical case;

the interior space of the cylindrical cap comprises a jacket for receiving the retractable needle shield when present; and

the interior space of the cylindrical cap comprises a locking projection for engaging with the autoinjector mechanism when present.

25. An autoinjector, comprising:

the autoinjector housing of item 1 or any item depending therefrom; and an autoinjector mechanism.

26. The autoinjector of item 25 or any item depending therefrom, wherein the autoinjector mechanism comprises:

a syringe filled with a single dose of a medicament; and

a retractable needle shield that surrounds a needle of the syringe;

wherein the retractable needle shield of the autoinjector is provided in the opening in the open injector end of the cylindrical case.

27. The autoinjector of item 25 or any item depending therefrom, wherein the autoinjector is a button-less autoinjector.

28. The autoinjector of item 25 or any item depending therefrom, wherein, after removing the cylindrical cap, the autoinjector can be operated one-handed.

29. The autoinjector of item 25 or any item depending therefrom, comprising a syringe filled with a single dose of adalimumab.

30. The autoinjector of item 29 or any item depending therefrom, wherein the single dose of adalimumab comprises 40 mg of adalimumab at a concentration of 50 mg/ml.

31. The autoinjector of item 29 or any item depending therefrom, wherein the single dose of adalimumab comprises 40 mg of adalimumab at a concentration of 100 mg/ml.

32. A method of treating a disease or disorder, comprising administering a single dose of a tumor necrosis factor (TNF) blocker to a patient in need of such treatment with the autoinjector of item 25 or any item depending therefrom, wherein the disease or disorder is at least one selected from the group consisting of rheumatoid arthritis, juvenile idiopathic arthritis, psoriatic arthritis, ankylosing spondylitis, Crohn's disease, ulcerative colitis, plaque psoriasis, hidradenitis suppurativa, and uveitis.

33. The method of item 32 or any item depending therefrom, wherein the tumor necrosis factor (TNF) blocker is adalimumab.

34. The method of item 32 or any item depending therefrom, where administering comprises self-administering by the patient.

35. A method of injecting a medicament using the autoinjector of item 26 or any item depending therefrom, comprising:

removing the cylindrical cap from the cylindrical case, if attached;

grasping the autoinjector;

positioning the autoinjector so the retractable needle shield is in contact with an injection site on a patient's skin and the first axis of cylindrical case is substantially perpendicular to the injection site;

pressing the autoinjector into the skin in a direction substantially perpendicular to the injection site to cause (i) the needle to insert into the injection site, (ii) the retractable needle shield to retract into the cylindrical case, and (iii) the single dose of the medicament to be injected into the injection site; and

pulling the autoinjector away from the injection site resulting in return of the retractable needle shield to an unretracted state when administration of the single dose of the medicament is complete.

36. The method of item 35 or any item depending therefrom, wherein, after removing the cylindrical cap, the method is performed one-handed.

37. The method of item 35 or any item depending therefrom, wherein the method is performed without pressing an actuator or button.

38. The method of claim 35, wherein the injection of the medicament is accompanied by at least one audible cue.

39. An autoinjector housing, comprising:

a cylindrical case enclosing an interior space capable of accommodating an autoinjector mechanism;

a cylindrical cap removably attachable to the cylindrical case; and

a cylindrical overmolding;

wherein:

the cylindrical case has a first axis extending from a closed handle end to an open injector end of the cylindrical case;

an exterior cross-sectional shape of the cylindrical case is oblong along at least a portion of the first axis;

the open injector end of the cylindrical case comprises an opening capable of accommodating a retractable needle shield of the autoinjector mechanism;

the cylindrical cap has a second axis extending from an open end to a closed end of the cylindrical cap;

an exterior cross-sectional shape of the cylindrical cap is oblong along at least a portion of the second axis;

the cylindrical cap is removably attached to the cylindrical case by fitting the open end of the cylindrical cap over the open injector end of the cylindrical case such that the first axis and second axis are substantially aligned;

the cylindrical overmolding has a third axis extending from a closed end to an open end of the cylindrical overmolding;

the cylindrical overmolding encloses an interior space accommodating at least a portion of the cylindrical case such that the third axis coincides with the first axis;

at least the closed handle end of the cylindrical case is covered by the cylindrical overmolding;

the cylindrical overmolding comprises a material having elasticity; and

the cylindrical overmolding has a non-uniform radial thickness measured in a direction perpendicular to the third axis.

40. The autoinjector housing of item 39 or any item depending therefrom, wherein:

an exterior cross-sectional shape of the cylindrical case is varied along the first axis such that there is a step-up in cross-sectional area along the first axis approaching the open injector end;

the step-up in cross-sectional area of the cylindrical case forms a overmolding flange, and the cylindrical overmolding covering covers the cylindrical case from the closed handle end to the overmolding flange; and

the exterior cross-sectional shape of the cylindrical case on an open-injector side of the overmolding flange and an exterior cross-sectional shape of the cylindrical overmolding covering at the open end are substantially the same.

41. The autoinjector housing of item 39 or any item depending therefrom, wherein:

the cylindrical overmolding comprises a plurality of openings extending through the cylindrical overmolding in a radial direction; and

when viewed from an exterior of the cylindrical overmolding, an exterior of the cylindrical case is visible through each of the plurality of openings.

42. The autoinjector housing of item 41 or any item depending therefrom, wherein the radial thickness of the cylindrical overmolding adjacent to and forming at least one of the openings is greater than an average radial thickness of the cylindrical overmolding.

43. The autoinjector housing of item 41 or any item depending therefrom, wherein

a portion of the cylindrical overmolding adjacent to and forming at least one of the openings comprises at least two sub-portions having different radial thicknesses.

44. The autoinjector housing of item 41 or any item depending therefrom, wherein

the radial thickness of the cylindrical overmolding adjacent to and forming a first one of the plurality of openings is greater than the radial thickness of the cylindrical overmolding adjacent to and forming a second one of the plurality of openings.

45. The autoinjector housing of item 41 or any item depending therefrom, wherein:

the plurality of openings comprises at least one grouping of openings arranged in a grid or matrix when viewed from an exterior of the cylindrical overmolding; and

the at least one grouping of openings extends over at least ten percent of an external surface area of the cylindrical overmolding.

46. The autoinjector housing of item 45 or any item depending therefrom, wherein each of the plurality of openings is polygonal in shape.

47. The autoinjector housing of item 45 or any item depending therefrom, wherein each of the plurality of openings is hexagonal in shape.

48. The autoinjector housing of item 45 or any item depending therefrom, wherein:

the plurality of openings comprises a first grouping of openings arranged in a grid or matrix when viewed from the exterior of the cylindrical overmolding and a second grouping of openings arranged in a grid or matrix when viewed from the exterior of the cylindrical overmolding; and

the first grouping of openings and the second grouping of openings are arranged on opposite sides of the third axis.

49. The autoinjector housing of item 48 or any item depending therefrom, wherein:

an exterior cross-sectional shape of the cylindrical overmolding is oblong along an entirety of the third axis; and

the first grouping of openings and the second grouping of openings are arranged on opposite sides of a longest dimension of cylindrical overmolding in a direction perpendicular to the third axis.

50. The autoinjector housing of item 49 or any item depending therefrom, wherein each of the groupings comprises a plurality of hexagonal shaped openings arranged in a honeycomb grid.

51. The autoinjector housing of item 41 or any item depending therefrom, wherein:

the plurality of openings comprises at least one grouping of openings arranged in a grid or matrix;

the grid or matrix comprises at least three adjacent rows of openings extending along the cylindrical overmolding in a circumferential direction, including at least one central row and at least two peripheral rows;

the grid or matrix comprises at least three adjacent columns of openings extending along the cylindrical overmolding in an axial direction, including at least one central column and at least two peripheral columns;

the radial thickness of at least a portion the cylindrical overmolding adjacent to and forming at least one of the openings in the central row is greater than the radial thickness of at least a portion the cylindrical overmolding adjacent to and forming at least one of the openings in at least one of the peripheral rows; and

the radial thickness of at least a portion the cylindrical overmolding adjacent to and forming at least one of the openings in the central column is greater than the radial thickness of at least a portion of the cylindrical overmolding adjacent to and forming at least one of the openings in at least one of the peripheral columns.

52. The autoinjector housing of item 39 or any item depending therefrom, wherein:

the cylindrical case comprises at least one window opening extending through the cylindrical case in a radial direction;

when the autoinjector mechanism is accommodated in the interior space and comprises a syringe, contents of the syringe can be seen through the window opening; and

the window opening has an oblong shape when viewed from an exterior of the cylindrical case with a major axis of the oblong shape being parallel to the first axis of the cylindrical case.

53. The autoinjector housing of item 39 or any item depending therefrom, wherein the cylindrical cap is the only cap of the autoinjector housing.

54. The autoinjector housing of item 39 or any item depending therefrom, wherein the housing does not comprise an opening for accommodating a button or actuator.

55. The autoinjector housing of item 39 or any item depending therefrom, wherein the cylindrical cap comprises a transparent or translucent material.

56. The autoinjector housing of item 39 or any item depending therefrom, wherein the cylindrical cap and the cylindrical overmolding differ in at least one surface characteristic.

57. The autoinjector housing of item 56 or any item depending therefrom, wherein the cylindrical cap and the cylindrical overmolding differ in color.

58. The autoinjector housing of item 56 or any item depending therefrom, wherein the cylindrical cap and the cylindrical overmolding differ in texture.

59. The autoinjector housing of item 39 or any item depending therefrom, wherein the cylindrical case, the cylindrical cap, and the cylindrical overmolding differ in at least one surface characteristic.

60. The autoinjector housing of item 59 or any item depending therefrom, wherein the cylindrical case, the cylindrical cap, and the cylindrical overmolding differ in color.

61. The autoinjector housing of item 59 or any item depending therefrom, wherein the cylindrical case, the cylindrical cap, and the cylindrical overmolding differ in texture.

62. An autoinjector, comprising:

the autoinjector housing of item 39 or any item depending therefrom; and

an autoinjector mechanism.

63. The autoinjector of item 62, wherein the autoinjector mechanism comprises:

a syringe filled with a single dose of a medicament; and

a retractable needle shield that surrounds a needle of the syringe;

wherein the retractable needle shield of the autoinjector is provided in the opening in the open injector end of the cylindrical case.

64. The autoinjector of item 62 or any item depending therefrom, wherein the autoinjector is a button-less autoinjector.

65. The autoinjector of item 62 or any item depending therefrom, wherein, after removing the cylindrical cap, the autoinjector can be operated one-handed.

66. The autoinjector of item 62 or any item depending therefrom, comprising a syringe filled with a single dose of adalimumab.

67. The autoinjector of item 66 or any item depending therefrom, wherein the single dose of adalimumab comprises 40 mg of adalimumab at a concentration of 50 mg/ml.

68. The autoinjector of item 66 or any item depending therefrom, wherein the single dose of adalimumab comprises 40 mg of adalimumab at a concentration of 100 mg/ml.

69. A method of treating a disease or disorder, comprising administering a single dose of a tumor necrosis factor (TNF) blocker to a patient in need of such treatment with the autoinjector of item 62 or any item depending therefrom, wherein the disease or disorder is at least one selected from the group consisting of rheumatoid arthritis, juvenile idiopathic arthritis, psoriatic arthritis, ankylosing spondylitis, Crohn's disease, ulcerative colitis, plaque psoriasis, hidradenitis suppurativa, and uveitis.

70. The method of item 69 or any item depending therefrom, wherein the tumor necrosis factor (TNF) blocker is adalimumab.

71. The method of item 69 or any item depending therefrom, where administering comprises self-administering by the patient.

72. A method of injecting a medicament using the autoinjector of item 63 or any item depending therefrom, comprising:

removing the cylindrical cap from the cylindrical case, if attached;

grasping the autoinjector;

positioning the autoinjector so the retractable needle shield is in contact with an injection site on a patient's skin and the first axis of cylindrical case is substantially perpendicular to the injection site;

pressing the autoinjector into the skin in a direction substantially perpendicular to the injection site to cause (i) the needle to insert into the injection site, (ii) the retractable needle shield to retract into the cylindrical case, and (iii) the single dose of the medicament to be injected into the injection site; and

pulling the autoinjector away from the injection site resulting in return of the retractable needle shield to an unretracted state when administration of the single dose of the medicament is complete.

73. The method of item 72 or any item depending therefrom, wherein, after removing the cylindrical cap, the method is performed one-handed.

74. The method of item 72 or any item depending therefrom, wherein the method is performed without pressing an actuator or button.

75. The method of item 72 or any item depending therefrom, wherein grasping the autoinjector comprises grasping the cylindrical overmolding.

76. The method of claim 72, wherein the injection of the medicament is accompanied by at least one audible cue.

The foregoing discussion discloses and describes merely exemplary embodiments of the present disclosure. As will be understood by those skilled in the art, the present disclosure may be embodied in other specific forms without departing from the spirit or essential characteristics thereof. Accordingly, the disclosure of the present invention is intended to be illustrative, but not limiting of the scope of the disclosure, as well as other claims. The disclosure, including any readily discernible variants of the teachings herein, defines, in part, the scope of the foregoing claim terminology such that no inventive subject matter is dedicated to the public.

1. An autoinjector housing, comprising:
a cylindrical case enclosing an interior space capable of accommodating an autoinjector mechanism; and
a cylindrical cap removably attachable to the cylindrical case;
wherein:
the cylindrical case has a first axis extending from a closed handle end to an open injector end of the cylindrical case;
an exterior cross-sectional shape of the cylindrical case is varied along the first axis such that there is a step-down in cross-sectional area along the first axis approaching the open injector end;
the exterior cross-sectional shape of the cylindrical case is oblong along at least a portion of the first axis;
the open injector end of the cylindrical case comprises an opening capable of accommodating a retractable needle shield of the autoinjector mechanism;
the cylindrical cap has a second axis extending from an open end to a closed end of the cylindrical cap;
an exterior cross-sectional shape of the cylindrical cap is varied along the second axis such that there is a gradual decrease in cross-sectional area from the open end toward the closed end and a step-up in cross-sectional area approaching the closed end;
the exterior cross-sectional shape of the cylindrical cap is oblong along at least a portion of the second axis;
the cylindrical cap is removably attached to the cylindrical case by fitting the open end of the cylindrical cap over the open injector end of the cylindrical case such that the first axis and second axis are substantially aligned; and
the step-down in cross-sectional area of the cylindrical case forms a cap flange, and attachment of the cylindrical cap is completed when the open end of the cylindrical cap is seated on the cap flange.

2. The autoinjector housing of claim 1, wherein:
the exterior cross-sectional shape of the cylindrical case is elliptical along at least a portion of the first axis; and
the exterior cross-sectional shape of the cylindrical cap is elliptical along at least a portion of the second axis.

3. The autoinjector housing of claim 2, wherein:
the exterior cross-sectional shape of the cylindrical case is elliptical for at least 50% of the length of the cylindrical case along the first axis; and
the exterior cross-sectional shape of the cylindrical cap is elliptical for at least 50% of the length of the cylindrical cap along the second axis.

4. The autoinjector housing of claim 2, wherein:
the elliptical exterior cross-sectional shape of the cylindrical case has an average ratio of major axis length to minor axis length of 1.1:1 to 2.5:1 along the first axis; and
the elliptical exterior cross-sectional shape of the cylindrical cap has a ratio of major axis length to minor axis length of 1.1:1 to 2.5:1 along the second axis.

5. The autoinjector housing of claim 1, wherein:
the oblong exterior cross-sectional shape of the cylindrical case along at least a portion of the first axis is selected from the group consisting of substantially oval, substantially hexagonal, and substantially octagonal; and
the oblong exterior cross-sectional shape of the cylindrical cap along at least a portion of the second axis is selected from the group consisting of substantially oval, substantially hexagonal, and substantially octagonal.

6. The autoinjector housing of claim 1, further comprising a cylindrical overmolding having a third axis extending from a closed end to an open end of the cylindrical overmolding; wherein:
the cylindrical overmolding encloses an interior space accommodating at least a portion of the cylindrical case such that the third axis coincides with the first axis;
at least the closed handle end of the cylindrical case is covered by the cylindrical overmolding; and
the cylindrical overmolding comprises a material having elasticity.

7. The autoinjector housing of claim 6, wherein the cylindrical overmolding covers at least one third of a length of the cylindrical case along the first axis extending from the closed handle end.

8. The autoinjector housing of claim 6, wherein the cylindrical cap and the cylindrical overmolding differ in at least one surface characteristic.

9. The autoinjector housing of claim 8, wherein the cylindrical cap and the cylindrical overmolding differ in color.

10. The autoinjector housing of claim 8, wherein the cylindrical cap and the cylindrical overmolding differ in texture.

11. An autoinjector housing, comprising:
a cylindrical case enclosing an interior space capable of accommodating an autoinjector mechanism;
a cylindrical cap removably attachable to the cylindrical case; and
a cylindrical overmolding;
wherein:
the cylindrical case has a first axis extending from a closed handle end to an open injector end of the cylindrical case;
an exterior cross-sectional shape of the cylindrical case is oblong along at least a portion of the first axis;
the open injector end of the cylindrical case comprises an opening capable of accommodating a retractable needle shield of the autoinjector mechanism;
the cylindrical cap has a second axis extending from an open end to a closed end of the cylindrical cap;
an exterior cross-sectional shape of the cylindrical cap is oblong along at least a portion of the second axis;
the cylindrical cap is removably attached to the cylindrical case by fitting the open end of the cylindrical cap over the open injector end of the cylindrical case such that the first axis and second axis are substantially aligned;
the cylindrical overmolding has a third axis extending from a closed end to an open end of the cylindrical overmolding;
the cylindrical overmolding encloses an interior space accommodating at least a portion of the cylindrical case such that the third axis coincides with the first axis;
at least the closed handle end of the cylindrical case is covered by the cylindrical overmolding;
the cylindrical overmolding comprises a material having elasticity; and
the cylindrical overmolding has a non-uniform radial thickness measured in a direction perpendicular to the third axis.

12. The autoinjector housing of claim 11, wherein:
an exterior cross-sectional shape of the cylindrical case is varied along the first axis such that there is a step-up in cross-sectional area along the first axis approaching the open injector end;
the step-up in cross-sectional area of the cylindrical case forms a overmolding flange, and the cylindrical overmolding covering covers the cylindrical case from the closed handle end to the overmolding flange; and
the exterior cross-sectional shape of the cylindrical case on an open-injector side of the overmolding flange and an exterior cross-sectional shape of the cylindrical overmolding covering at the open end are substantially the same.

13. The autoinjector housing of claim 11, wherein:
the cylindrical overmolding comprises a plurality of openings extending through the cylindrical overmolding in a radial direction; and
when viewed from an exterior of the cylindrical overmolding, an exterior of the cylindrical case is visible through each of the plurality of openings.

14. The autoinjector housing of claim 13, wherein the radial thickness of the cylindrical overmolding adjacent to and forming at least one of the openings is greater than an average radial thickness of the cylindrical overmolding.

15. The autoinjector housing of claim 13, wherein
a portion of the cylindrical overmolding adjacent to and forming at least one of the openings comprises at least two sub-portions having different radial thicknesses.

16. The autoinjector housing of claim 13, wherein
the radial thickness of the cylindrical overmolding adjacent to and forming a first one of the plurality of openings is greater than the radial thickness of the cylindrical overmolding adjacent to and forming a second one of the plurality of openings.

17. The autoinjector housing of claim 13, wherein:
the plurality of openings comprises at least one grouping of openings arranged in a grid or matrix when viewed from an exterior of the cylindrical overmolding; and
the at least one grouping of openings extends over at least ten percent of an external surface area of the cylindrical overmolding.

18. The autoinjector housing of claim 17, wherein each of the plurality of openings is polygonal in shape.

19. The autoinjector housing of claim 17, wherein each of the plurality of openings is hexagonal in shape.

20. The autoinjector housing of claim 17, wherein:
the plurality of openings comprises a first grouping of openings arranged in a grid or matrix when viewed from the exterior of the cylindrical overmolding and a second grouping of openings arranged in a grid or matrix when viewed from the exterior of the cylindrical overmolding; and
the first grouping of openings and the second grouping of openings are arranged on opposite sides of the third axis.
